(19) United States
(12) Patent Application Publication
(10) Pub. No.: US 2002/0115092 A1
(43) Pub. Date: Aug. 22, 2002

(54) Energy transfer labels with mechanically linked fluorophores

(75) Inventor: Julius Rebek JR., La Jolla, CA (US)

Correspondence Address: Kevin J. Forrestal, FOLEY & LARDNER, P.O. Box 80278, San Diego, CA, 92138-0278, US

(73) Assignee: The Scripps Research Institute

(21) Appl. No.: 10/005,987

(22) Filed: Nov. 8, 2001

Related U.S. Application Data
(60) Provisional application No. 60/247,522, filed on Nov. 8, 2000.

(51) Int. Cl. C12Q 1/68; C07H 21/04; C07D 311/88

(52) U.S. Cl. 435/6; 536/23.2; 549/225

(57) ABSTRACT

Mechanically linked energy transfer labels comprising at least one donor fluorophore, at least one acceptor fluorophore, and at least one support member, wherein steric interactions between the donor fluorophore(s), the acceptor fluorophore(s), and/or the support member(s) induce non-covalent association between the fluorophores and the support member(s), thereby forming a three-dimensional macromolecular structure which mechanically links the donor fluorophore(s) and the acceptor fluorophore(s). Fluorescence resonance energy transfer (FRET) occurs from donor fluorophore to acceptor fluorophore through space. No direct connectivity with covalent bonds exists between the fluorophores. Instead, mechanical barriers hold the donor/acceptor fluorophores in place during the FRET process.

### RELATED APPLICATIONS

This application is a continuation-in-part of U.S. Ser. No. 60/247,522.

This invention was made with Government support under Grant No. GM 27932 from the U.S. National Institutes of Health. The Government has certain rights to this invention.

### FIELD OF INVENTION

The present invention relates to energy transfer labels and methods for use thereof.

### BACKGROUND OF THE INVENTION

The following discussion of the background of the invention is merely provided to aid the reader in understanding the invention and is not admitted to describe or constitute prior art to the present invention.

Energy transfer labels are widely used in qualitative and quantitative analytical biology. Biological applications of energy transfer labels typically involve the transfer and emission of fluorescent energy, primarily due to the inherently increased sensitivity of fluorescence spectroscopy relative to absorption spectroscopy. Fluorescence resonance energy transfer labels have been used extensively to identify and detect a variety of biologically active molecules (e.g., nucleic acids, oligonucleotides, proteins).

Fluorescence resonance energy transfer (FRET) is a process by which an excited species (donor) transfers some of its energy to another species (acceptor). Fluorescence resonance energy transfer labels contain at least one donor fluorophore and at least one acceptor fluorophore. Each fluorophore must meet certain requirements in order to be employed as a component of a fluorescence resonance energy transfer label. For example, the donor fluorophore must absorb excitation energy and transfer some of this energy to the acceptor fluorophore. In turn, the acceptor fluorophore must absorb some of the energy transferred by the donor fluorophore and subsequently emit some of that energy at a longer maximum wavelength than that used to excite the donor fluorophore. A donor fluorophore, an acceptor fluorophore, and a component that connects the two fluorophores constitute a fluorescence resonance energy transfer label.

Currently the most common use of fluorescence resonance energy transfer labels is in DNA sequencing. Typically, a single donor fluorophore is used in conjunction with a variety of acceptor fluorophores in extension reactions terminated with dideoxyadenine, dideoxythymine, dideoxyguanosine and dideoxycytosine.

### SUMMARY OF THE INVENTION

In accordance with the present invention, there are provided mechanically linked energy transfer labels having at least one donor fluorophore, at least one acceptor fluorophore, and at least one support member. Energy transfer labels according to the present invention are useful in identifying and detecting a variety biologically active molecules (e.g., nucleic acids, oligonucleotides, proteins).

In a first aspect, there are provided mechanically linked energy transfer labels having at least one donor fluorophore, at least one acceptor fluorophore, and at least one support member, wherein steric interactions between the donor fluorophore(s), the acceptor fluorophore(s), and/or the support member(s) induce non-covalent association between the fluorophores and the support member(s), thereby forming a macromolecular structure which mechanically links the donor fluorophore(s) and the acceptor fluorophore(s). No direct connectivity with covalent bonds exists between the fluorophores. Instead, mechanical barriers hold the donor/acceptor fluorophores in place during the FRET process.

As used herein, the phrase “mechanically linked” refers to an interaction between donor fluorophore(s), acceptor fluorophore(s), and support member(s), wherein the donor fluorophore(s) and acceptor fluorophore(s) are not directly linked to each other with covalent bonds, and wherein the interaction results in fluorescence resonance energy transfer between donor fluorophore(s) and acceptor fluorophore(s). The term is not intended to refer to incorporation of donor and acceptor fluorophores individually into particles, as described in, e.g., U.S. Pat. No. 6,238,931, but rather to a physical, noncovalent linkage between donor and acceptor fluorophores.

As used herein, “fluorescence resonance energy transfer” refers to a process by which donor and acceptor fluorophores are functionally linked such that the donor-acceptor pair exhibits an absorbance peak corresponding to absorbance by the donor fluorophore, but in which at least some of the absorbed energy that would be emitted as light photons by the donor fluorophore in the absence of the acceptor fluorophore is reduced, or “quenched.” The donor-acceptor pair also exhibits an emission peak corresponding emission by the acceptor fluorophore.

While fluorescence energy transfer is described below in reference to a single donor and a single acceptor, the skilled artisan will understand that several fluorophores may be combined in series, where, for example, a first fluorophore acts as a donor to a second fluorophore, which itself acts as a donor to a third fluorophore. Alternatively, a fluorescence energy transfer system may comprise multiple donor fluorophores coupled to a single acceptor fluorophore, or multiple acceptor fluorophores coupled to a single donor fluorophore.

Fluorescence energy transfer is measured by exciting the donor-acceptor pair at the peak absorbance wavelength exhibited by the donor fluorophore alone, and measuring emissions at the peak emission wavelengths exhibited by the donor fluorophore and by the acceptor fluorophore. This is then compared to peak emission by the donor fluorophore in the absence of acceptor, and of the acceptor fluorophore in the absence of donor, when each is excited at the peak absorbance wavelength of the donor fluorophore. While fluorescence energy transfer as used herein does not require that all of the light emission by the donor is quenched, in preferred embodiments, at least 50% of the light emission is quenched, more preferably 75% is quenched, even more preferably 90% is quenched, and most preferably, at least 97% is quenched. Similarly, while fluorescence energy transfer as used herein does not require that the light emitted by the acceptor be increased relative to that observed from the donor alone, in preferred embodiments emission from the donor is increased by at least 10%, more preferably at least 50%, even more preferably at least 100%, and most preferably at least 200%. Preferred are those fluorescence energy transfer systems in which at least 90% of the emitted light is produced at wavelengths corresponding to emission by the acceptor fluorophore, and most preferred are those in which at least 95% of the emitted light is produced at wavelengths corresponding to emission by the acceptor fluorophore.

As used herein, the term “donor fluorophore” refers to a moiety in a fluorescence energy transfer system which absorbs energy, and which exhibits a quenched photonic emission relative to that exhibited by the same fluorophore alone.

As used herein, the term “acceptor fluorophore” refers to a moiety in a fluorescence energy transfer system which exhibits a maximum photonic emission wavelength greater than that of a donor fluorophore in the system.

As used herein, the phrase “support member” refers to any molecule (e.g., organic) to which the donor and acceptor fluorophores are covalently attached or non-covalently associated via steric interactions.

As used herein, the phrase “non-covalent association” refers to an arrangement wherein the support members are assembled via steric interactions, i.e., the structural integrity of the arrangement does not rely on covalent bonding interactions between individual support members.

As used herein, the phrase “steric interactions” refers to relationships between support members which are defined by the three-dimensional shape of each support member (e.g., the molecular Van der Waals' radii of each support member), and are not dependent on electronic bonding interactions (e.g., covalent bonding).

The support members non-covalently associate with each other and with one or more fluorophores to form macromolecular assemblies, such as, for example, rotaxanes, catenanes, carcerands, hemicarcerands, resorcinarenes, calixarene capsules.

In one embodiment of the present invention, the energy transfer labels contain two support members. The fluorophores and the biomolecule may be covalently attached to the support members or non-covalently associated with the support members. In a preferred aspect of this embodiment, a donor fluorophore is covalently attached to a first support member and an acceptor fluorophore is covalently attached to a second support member. In an especially preferred aspect of this embodiment, a first support member interacts sterically with a second support member to form a rotaxane, thereby mechanically linking the fluorophores. As used herein, the term “rotaxane” refers to a macromolecular structure having a linear molecule (molecular axle) threaded through a macrocycle ( molecular wheel). This structure is analogous to a ring positioned around a bone (or dumbbell), where movement of the ring over the bone (or dumbbell) occurs freely, but the ring can not be easily removed from the ends of the bone (or dumbbell) (see FIG. 1B). However, under certain conditions it is possible to alter the steric interactions between the ring and the bone so that the ring can be removed from the bone.

As used herein, the phrase “linear molecule” refers to any molecule which can be inserted into a macrocycle.

As used herein, the phrase “macrocycle” refers to a circular molecule with a diameter of a suitable size to allow for insertion of a linear molecule.

Energy transfer labels having a rotaxane-type assembly comprise molecular axles having the structure:

St-L-St,

wherein:

L is hydrocarbyl linking moiety, and

St is a stopper moiety capable of being covalently attached to said linking moiety and at least one donor or acceptor fluorophore.

As employed herein, the term “hydrocarbyl” refers to a moiety formed from hydrogen and carbon, e.g., alkyl, substituted alkyl, cycloalkyl, substituted cycloalkyl, alkenyl, substituted alkenyl, alkynyl, substituted alkynyl, aryl, substituted aryl.

As employed herein, “alkyl” refers to hydrocarbyl radicals having 1 up to 20 carbon atoms, or any subset thereof, preferably 2-10 carbon atoms; and “substituted alkyl” comprises alkyl groups further bearing one or more substituents selected from hydroxy, alkoxy, mercapto, cycloalkyl, substituted cycloalkyl, heterocyclic, substituted heterocyclic, aryl, substituted aryl, heteroaryl, substituted heteroaryl, aryloxy, substituted aryloxy, halogen, cyano, nitro, amino, amido, C(O)H, acyl, oxyacyl, carboxyl, carbamate, sulfonyl, sulfonamide, sulfuryl.

As employed herein, “cycloalkyl” refers to cyclic ring-containing groups containing in the range of about 3 up to 8 carbon atoms, or any subset thereof, and “substituted cycloalkyl” refers to cycloalkyl groups further bearing one or more substituents as set forth above.

As employed herein, “alkenyl” refers to straight or branched chain hydrocarbyl groups having at least one carbon-carbon double bond, and having in the range of about 2 up to 12 carbon atoms, or any subset thereof, and “substituted alkenyl” refers to alkenyl groups further bearing one or more substituents as set forth above.

As employed herein, “alkynyl” refers to straight or branched chain hydrocarbyl groups having at least one carbon-carbon triple bond, and having in the range of about 2 up to 12 carbon atoms, or any subset thereof, and “substituted alkynyl” refers to alkynylene groups further bearing one or more substituents as set forth above.

As employed herein, “aryl” refers to aromatic groups having in the range of 6 up to about 14 carbon atoms, or any subset thereof, and “substituted aryl” refers to aryl groups further bearing one or more substituents as set forth above.

In one aspect of this embodiment, the hydrocarbyl linking moiety comprises at least one aryl group. In a preferred aspect of this embodiment, the hydrocarbyl linking moiety comprises at least two aryl groups. In an especially preferred aspect of this embodiment, the two aryl groups are separated by an optionally substituted C₁ to C₆ alkyl group or heteroalkyl group. As used herein, “heteroalkyl” refers to an alkyl group wherein one or more of the carbon atoms in the alkyl group are replaced with heteroatoms. As used herein, “heteroatom” refers to N, O, S, or P.

As used herein, the phrase “stopper moiety” refers to a moiety which, in a rotaxane assembly, prevents via steric hindrance the linear molecular axle from slipping out of the macrocycle wheel. Preferred stopper moieties include substituted cyclic moieties such as, for example, cycloaliphatic, heterocyclic, aryl, heteroaryl groups. Preferred substituents on these cyclic moieties include, for example, hydroxyl, amine, carboxyl, amide, hydroxyalkyl, aminoalkyl.

Energy transfer labels having a rotaxane-type assembly employ macrocycles for use as molecular wheels, wherein the macrocycle is capable of being covalently attached to at least one donor or acceptor fluorophore and is capable of being attached to a biomolecule. As used herein, the word “biomolecule” refers to nucleosides, nucleotides, oligonucleotides, polynucleotides, proteins, and polysaccharides. Suitable functional groups for attaching a fluorophore to a macrocycle include, for example, hydroxyl, carboxyl, amino, amido, thio.

Macrocycles contemplated for use in the practice of the present invention comprise subunits linked in a cyclic manner. Subunits contemplated for use in the practice of the present invention include optionally substituted alkyl, cycloalkyl, oxyalkyl, aryl, heteroaryl, heterocyclic. In a preferred aspect, the macrocycle comprises optionally substituted aryl or heteroaryl subunits. The monomers are linked in a cyclic manner either directly or via substituents which are optionally attached to the subunits. Substituents contemplated for use in the practice of the present invention include alkyl, amide, carboxyl, hydroxy, hydroxyalkyl, oxyalkyl, amino, alkylamino.

In another aspect, the macrocycle comprises optionally substituted oxyalkyl moieties, such as, for example, a crown ether.

In a further aspect of the invention wherein energy transfer labels contain two support members, the support members are physically interlocked, thereby mechanically linking the donor fluorophore(s) and acceptor fluorophore(s). As used herein, the phrase “physically interlocked” refers to a molecular arrangement wherein the support members can not be separated without breaking covalent bonds.

In a preferred aspect of this embodiment, each of the physically interlocked support members is a macrocycle, thereby forming a catenane assembly (see FIG. 1B). Each macrocycle is capable of being covalently attached to at least one donor or acceptor fluorophore and is capable of being attached to a biomolecule. Macrocycles contemplated for use in a catenane assembly contain subunits linked in a cyclic manner. Subunits contemplated for use in the practice of the present invention include substituted alkyl, cycloalkyl, oxyalkyl, aryl, heteroaryl, heterocyclic. In a preferred aspect, the macrocycle comprises optionally substituted aryl or heteroaryl subunits. The subunits are linked in a cyclic manner either directly or via substituents which are optionally attached to the subunits. Substituents contemplated for use in the practice of the present invention include alkyl, amide, carboxyl, hydroxy, hydroxyalkyl, oxyalkyl, amino, alkylamino.

In a still further embodiment of the present invention, energy transfer labels contain one support member capable of encapsulating one or more of the donor fluorophore, acceptor fluorophore, or biomolecule. As used herein, the word “encapsulate” refers to a situation wherein one or more of the donor fluorophore, acceptor fluorophore, or biomolecule is located entirely within an interior cavity of a single support member. The donor fluorophore, acceptor fluorophore, or biomolecule may also be covalently attached to this single support member. In one aspect, the single support member has a globular shape, wherein at least one component of the energy transfer label (i.e., donor fluorophore or acceptor fluorophore) is encapsulated within the globe, and a biomolecule is attached to the outside surface of the globe.

In a preferred aspect of this embodiment of the invention, the support member is a carcerand, wherein a donor fluorophore or acceptor fluorophore is entirely encapsulated within the carcerand. In this aspect of the invention, the encapsulated fluorophore can not escape the carcerand without breaking covalent bond(s) which form the carcerand structure. Carcerands contemplated for use in the practice of the present invention may be prepared in a number of ways, such as for example, by the method of Cram, D. J., et. al., J. Am. Chem. Soc., 1991, 113, 2167, the entire contents of which are incorporated by reference herein. Alternatively, the single support member is a hemicarcerand, wherein an encapsulated fluorophore can escape the interior of the hemicarcerand by thermally overcoming steric constraints imposed by the size and shape of the fluorophore and the hemicarcerand. Hemicarcerands contemplated for use in the practice of the present invention may be prepared in a number of ways, such as for example, by the method of Cram, D. J., et. al., J. Am. Chem. Soc., 1991, 113, 2754, the entire contents of which are incorporated by reference herein.

In a still further aspect of this embodiment of the invention, the single support member is a calixarene or resorcinarene. These are bowl-shaped molecules which can ensnare a fluorophore within the bowl-shaped interior, while simultaneously associating with another fluorophore via appropriate functionality on the outer rim of the bowl. Calixarenes and resorcinarenes contemplated for use in the practice of the present invention may be prepared in a number of ways, such as for example, via condensation reactions with suitable spacers, as described previously (see, Cram, et. al., J. Amer. Chem. Soc. 1991, 113:2194-2204, the entire contents of which are incorporated by reference herein).

A wide variety of fluorophores is contemplated for use in the practice of the present invention, such as, for example, xanthenes (e.g., fluoresceins, rhodamines), coumarins (e.g., umbelliferone), benzimides, phenanthridines (e.g., Texas Red), ethidium fluorophores, acridines, cyanines, phthalocyanines, squarines, carbazoles, phenoxazines, porphyrins, quinolines, and the like. In a preferred aspect, the fluorophores are xanthenes or coumarins. The fluorophores may absorb in the ultraviolet, visible, or infrared ranges of the electromagnetic spectrum.

In accordance with another aspect of the invention, there are provided methods for labeling a biomolecule comprising contacting the biomolecule with an energy transfer label under conditions suitable to form a covalent bond between the biomolecule and the energy transfer label, thereby forming a labeled biomolecule, wherein the energy transfer label comprises at least one donor fluorophore covalently attached to a first support member and at least one acceptor fluorophore covalently attached to a second support member, wherein steric interactions between the support members mechanically link the donor fluorophore and the acceptor fluorophore.

Fluorescence energy transfer labels may be attached covalently to a wide variety of biomolecules to form bioconjugates. Biomolecules contemplated for use as components of bioconjugates include, for example, nucleosides, nucleotides, oligonucleotides, polynucleotides, proteins, and polysaccharides. In one aspect, the biomolecule is preferably an oligonucleotide or a polynucleotide. Energy transfer labels may be attached to oligonucleotides at the 5′-terminus, the 3′-terminus, or on the phosphodiester backbone. Bioconjugates are useful in applications such as, for example, oligonucleotide hybridization probes, PCR primers, and DNA sequencing.

Fluorescence energy transfer labels are suitable for use in a wide variety of applications, both qualitative and quantitative, such as DNA sequencing and ligand-receptor assays. (see for example, Lee, et. al., U.S. Pat. No. 5,800,996, Mathies, et. al., U.S. Pat. No. 5,688,648, Buechler, et. al., U.S. Pat. No. 6,251,687, the entire contents of each are incorporated herein by reference). For example, energy transfer labels are suitable for identifying nucleic acids in a multi-nucleic acid mixture. In particular, energy transfer labels are useful in DNA sequencing. DNA sequencing involves extension and termination reactions of oligonucleotide primers. Included as components of the extension and termination reactions are deoxynucleoside triphosphates (dNTP's) and dideoxynucleoside triphosphates (ddNTP's); dNTP's are used to extend the primer and ddNTP's terminate further extension of the primer. The different termination products that are formed are separated and analyzed in order to determine the positioning of the various nucleosides.

Fluorescence energy transfer labels may be used to label oligonucleotide primers, dNTP's, or ddNTP's. Thus, in accordance with another aspect of the invention there are provided methods for DNA primer sequencing and DNA terminator sequencing. In DNA primer sequencing, the fluorescence energy transfer label is attached to the primer being extended. Four separate extension/termination reactions are then carried out simultaneously, each extension reaction containing a different ddNTP to terminate the extension reaction. After termination, the reaction products are separated by gel electrophoresis and analyzed. Thus, in accordance with this aspect of the invention, there is provided a method for sequencing a polynucleotide comprising forming a mixture of extended labeled primers by hybridizing a polynucleotide with an oligonucleotide primer labeled with an energy transfer label in the presence of deoxynucleoside triphosphates, at least one dideoxynucleoside triphosphate, and a DNA polymerase, wherein the DNA polymerase extends the primer with the deoxynucleoside triphosphates until a dideoxynucleoside triphosphate is incorporated which terminates extension of the primer, separating the mixture of extended labeled primers, and determining the sequence of the polynucleotide by irradiating the mixture of extended labeled primers.

In accordance with a still further aspect of the invention, there is provided a method for sequencing a polynucleotide comprising forming a mixture of extended primers by hybridizing a polynucleotide with an oligonucleotide primer in the presence of deoxynucleoside triphosphates, at least one dideoxynucleoside triphosphate labeled with an energy transfer label having mechanically linked fluorophores, and a DNA polymerase, wherein the DNA polymerase extends the primer with the deoxynucleoside triphosphates until a labeled dideoxynucleoside triphosphate is incorporated which terminates extension of the primer, separating the mixture of extended primers, and determining the sequence of the polynucleotide by detecting the labeled dideoxynucleoside triphosphate attached to the extended primers.

In accordance with yet another aspect of the invention, there is provided a method for sequencing a polynucleotide comprising forming a mixture of extended primers by hybridizing a polynucleotide with an oligonucleotide primer in the presence of deoxynucleoside triphosphates labeled with an energy transfer label having mechanically linked fluorophores, at least one dideoxynucleoside triphosphate, and a DNA polymerase, wherein the DNA polymerase extends the primer with the labeled deoxynucleoside triphosphates until a dideoxynucleoside triphosphate is incorporated which terminates extension of the primer, separating the mixture of extended primers, and determining the sequence of the polynucleotide by detecting the labeled deoxynucleoside triphosphates attached to the extended primers.

In accordance with another aspect of the invention, there is provided a method for increasing the intensity of a fluorescence resonance energy transfer signal comprising contacting an analyte with an energy transfer label having mechanically linked fluorophores under conditions suitable to form a covalent bond between said analyte and said energy transfer label, thereby forming a labeled analyte, irradiating said analyte at a first wavelength, and detecting energy emission at wavelengths other than said first wavelength.

Fluorescent energy transfer labels containing mechanical linking moieties (such as, for example, rotaxanes, catenanes, carcerands, hemicarcerands, calixarenes, resorcinarenes) which non-covalently link the fluorophores to each other and to the biomolecule of interest present an attractive alternative to the presently available labels containing covalent linkages. A mechanical linking moiety allows for increased control over the three-dimensional orientation of each fluorophore with respect to the other, thereby resulting in increased control over signal intensity and resolution.

Indeed, the orientation in space of each fluorophore is chosen to maximize energy transfer from donor fluorophore to acceptor fluorophore. Energy transfer is dependent on 1/R6, wherein R is the distance between the two fluorophores. In addition, the geometrical orientation of the dipoles of the donor and acceptor fluorophores will affect the efficiency of energy transfer between them (see, for example, Förster, Ann. Physik. (1948) 2, 55-75; Principles of Photochemistry, J. A. Baltrop and J. D. Coyle, 1978, page 118). In the present invention, appropriate spacing can be provided between the two fluorophores by suitable choice of support member(s) and three-dimensional macromolecular architecture (e.g., rotaxane, resorcinarene, catenane, carcerand, hemicarcerand, resorcinarene, calixarene) to which the fluorophores are either covalently attached or associated non-covalently via steric interactions. Mechanical barriers unique to each macromolecular assembly position the fluorophores properly to allow FRET to take place. Thus, the relative orientation of each fluorophore can be readily varied to optimize the signal produced by invention energy transfer labels during the FRET process.

### BRIEF DESCRIPTION OF THE FIGURES

FIG. 1A illustrates energy transfer labels having covalently linked fluorophores. The PE Biosystems “Big Dye” label is described in U.S. Pat. No. 5,800,996. The Amersham label is described in U.S. Pat. No. 5,688,648.

FIG. 1B schematically illustrates a rotaxane and a catenane.

FIG. 1C schematically illustrates an embodiment of the invention for the rotaxane type energy transfer label with mechanically linked fluorophores.

FIG. 2 illustrates a synthetic route to linear molecule (“axle”) 4 (for use in a rotaxane assembly) from a trans-stilbene dimethyl ester.

FIG. 3 illustrates a synthetic route to macrocycle (“wheel”) 9 for use in a first generation rotaxane assembly.

FIG. 4 illustrates a synthetic route for attaching an acceptor fluorophore to wheel 9, resulting in wheel 10.

FIGS. 5 and 6 illustrate a synthetic route to stopper 18 for use with a first generation rotaxane assembly.

FIG. 7 illustrates the reaction conditions under which stopper 18 is attached to axle 4 of the rotaxane.

FIG. 8 illustrates the rotaxane structure obtained when threading of wheel 10 occurs with stopper 18 attached to axle 4.

FIG. 9 illustrates the completed rotaxane where intermediate 19′ reacts with stopper 18′ to fix the wheel on the axle.

FIG. 10A shows two molecules that make up a second generation rotaxane with the donor fluorophore (dye₁) attached to the linear molecule (“axle”) and the acceptor fluorophore (dye₂) attached to the macrocycle (“wheel”).

FIG. 10B illustrates a further example of an unthreaded rotaxane type energy transfer label. Mechanical linkage of the fluorophores is achieved by threading the molecular “axle” through the molecular “wheel.”

FIG. 11 illustrates two molecules that make up an amino acid catenane.

FIG. 12A illustrates a deprotection step of a primary amine attached to one of the catenane rings

FIG. 12B illustrates a catenation scheme for two macrocycles.

FIG. 13 illustrates an expeditious synthesis of ester 103 from diester acid 101.

FIG. 14 illustrates a synthetic route to wheel 109.

FIG. 15 illustrates a synthetic route used to attach a diamine linker and a coumarin fluorophore 113 to the crown ether wheel 109, to form wheel 112.

FIG. 16 illustrates a synthetic route used attach a dideoxynucleoside to wheel 119, to form dideoxynucleoside functionalized wheel 120.

FIG. 17 illustrates a synthetic route used to prepare dideoxynucleotide functionalized wheel 121.

FIG. 18 illustrates the attachment of a 3′-hydroxy deprotected single strand of DNA to the 5′-triphosphate wheel 121 to afford wheel 123.

FIG. 19 illustrates the fluorescence spectrum of rotaxane 20 overlapped with the fluorescence spectrum of a mixture of stopper 18 and macrocycle 10.

### DETAILED DESCRIPTION OF THE INVENTION

In accordance with the present invention, there are provided energy transfer labels having at least one donor fluorophore, at least one acceptor fluorophore, and at least one support member mechanically linked via steric interactions between the fluorophores and the support member(s). The support members cooperatively associate with each other and with one or more fluorophores to form three-dimensional macromolecular assemblies, such as, for example, rotaxanes, catenanes, carcerands, hemicarcerands, calixarenes, resorcinarenes.

In one embodiment of the present invention, the energy transfer labels contain two support members. In a preferred aspect of this embodiment, a donor fluorophore is covalently attached to a first support member and an acceptor fluorophore is covalently attached to a second support member. In an especially preferred aspect of this embodiment, a first support member interacts sterically with a second support member to form a rotaxane. As used herein, the term “rotaxane” refers to a macromolecular structure having a linear molecule threaded through a macrocycle.

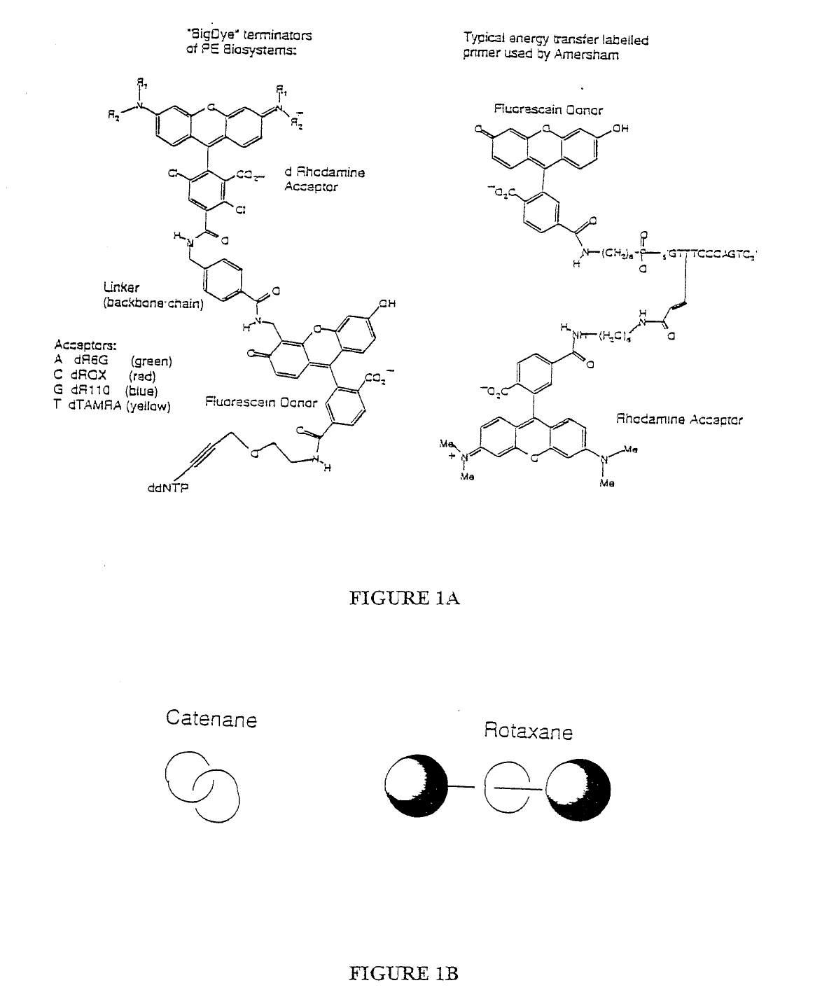

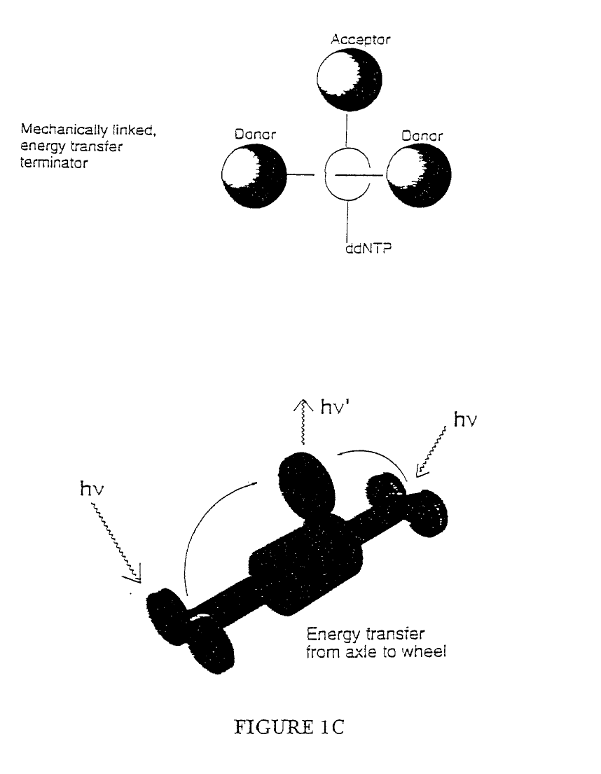

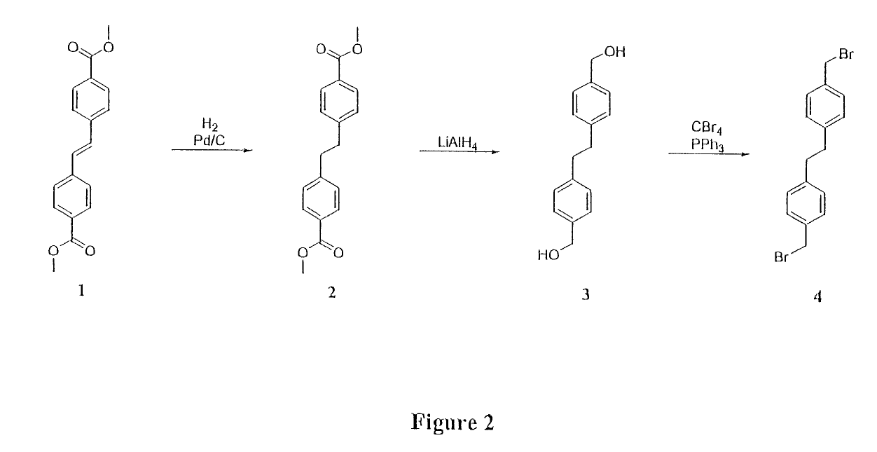

Rotaxane type fluorescent energy transfer labels are illustrated schematically in FIG. 1C. A wide variety of linear molecules (axles) and macrocycles (wheels) may be used to construct a rotaxane assembly suitable for use in the practice of the present invention (see, for example, Gibson, et. al., Macromolecules, 1997, 30(26); Raymo, et. al., Chem. Rev. 1999, 99, 1643, and references cited therein). The formation of a rotaxane structure from a linear molecular axle and a macrocyclic wheel may be confirmed by standard spectroscopic techniques, such as multi-nuclear NMR spectroscopy.

In a further aspect of the invention wherein energy transfer labels contain two support members, the support members are physically interlocked, thereby mechanically linking the donor fluorophore(s) and acceptor fluorophore(s). In a preferred aspect of this embodiment, each of the physically interlocked support members is a macrocycle, thereby forming a catenane assembly (see FIG. 1B).

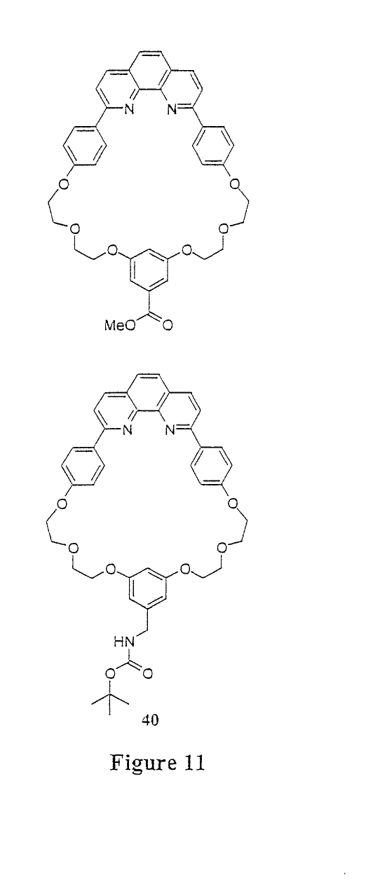

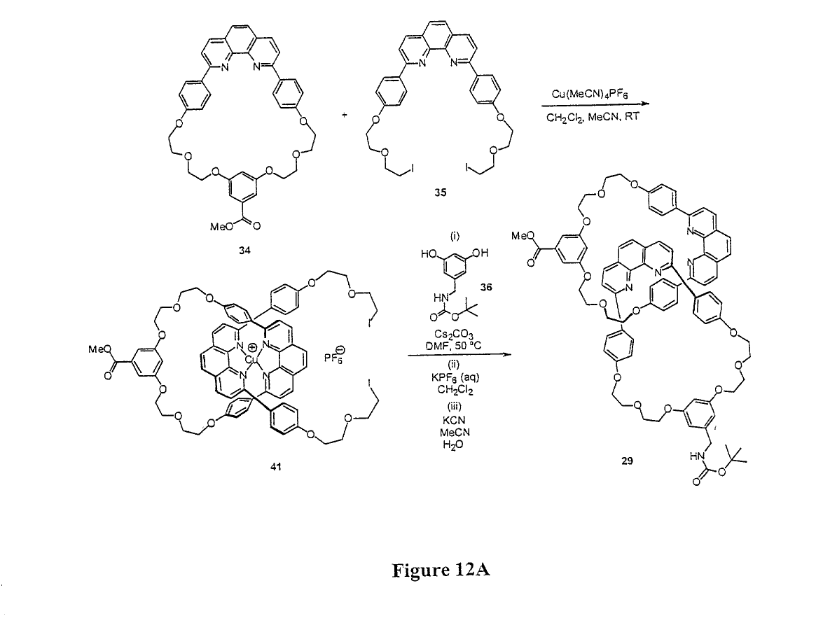

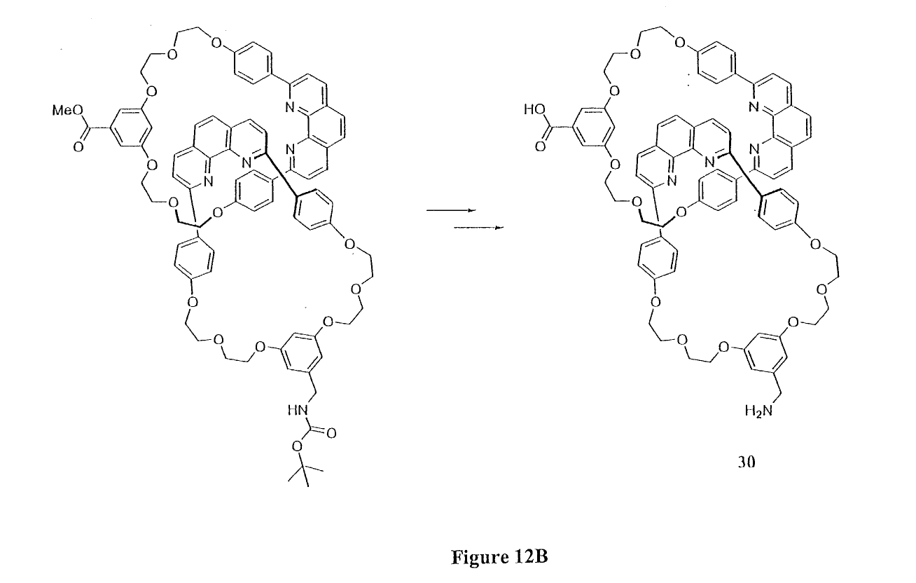

The preparation of macrocycles suitable for use in constructing a catenane assembly are well-known (see, for example, Pakula, et. al., Macromolecules, 1999, 32(20), 6821; Geerts, et. al., Macromolecules, 1999, 32(6), 1737; Stoddart, et. al., Macromolecules, 1998, 31(2), 295; the entire contents of each of which are incorporated by reference in their entirety). A catenane type fluorescent energy transfer label is synthesized by attaching fluorophores to the macrocycles via appropriate functionality, such as, for example, hydroxyl, carboxyl, amino, amide, thio. A catenane type fluorescent energy transfer label is illustrated in FIGS. 11, 12A and 12B. One macrocycle of the catenane bears an acid functional group and the other bears an amine. Referring to FIG. 12A, an exemplary catenation reaction was carried out according to Dietrich-Buchecker, C., et. al., Tetrahedron 1990, 46, 503, and Amabilino, D. B., et. al., New J Chem. 1998, 22, 395, the entire contents of each of which are incorporated by reference in their entirety. Confirmation of the catenane structure is typically provided by multi-nuclear NMR spectroscopy.

In a further aspect of the invention, the energy transfer labels have a single support member, wherein the fluorophores and/or the biomolecules are either encapsulated entirely within the support member or attached to the outer surface of the support member.

In a preferred aspect of this embodiment of the invention, the support member is a carcerand, wherein a donor fluorophore or acceptor fluorophore is entirely encapsulated within the carcerand. In this aspect of the invention, the encapsulated fluorophore can not escape the carcerand without breaking covalent bond(s) which form the carcerand structure. Carcerands contemplated for use in the practice of the present invention may be prepared in a number of ways, such as for example, by the method of Cram, D. J., et. al., J. Am. Chem. Soc., 1991, 113, 2167, the entire contents of which are incorporated by reference herein. Alternatively, the single support member is a hemicarcerand, wherein an encapsulated fluorophore can escape the interior of the hemicarcerand by thermally overcoming steric constraints imposed by the size and shape of the fluorophore and the hemicarcerand. Hemicarcerands contemplated for use in the practice of the present invention may be prepared in a number of ways, such as for example, by the method of Cram, D. J., et. al., J. Am. Chem. Soc., 1991, 113, 2754, the entire contents of which are incorporated by reference herein.

In a still further aspect of this embodiment of the invention, the single support member is a calixarene or resorcinarene. These are bowl-shaped molecules which can ensnare a fluorophore within the bowl-shaped interior, while simultaneously associating with another fluorophore via appropriate functionality on the outer rim of the bowl. Calixarenes and resorcinarenes contemplated for use in the practice of the present invention may be prepared in a number of ways, such as for example, via condensation reactions with suitable spacers, as described previously (see, Cram, et. al., J. Amer. Chem. Soc. 1991, 113:2194-2204, the entire contents of which are incorporated by reference herein). During the last step of the synthesis a donor/acceptor fluorophore capable of filling the interior of the bowl is present.

In another aspect, the labeled resorcinarene is connected to a hemicarcerand (see, Cram, et. al., J. Am. Chem. Soc., 1991, 113, 7717-7727, the entire contents of which are incorporated by reference herein). The resulting structure is used to surround the donor/acceptor.

In still another aspect of this embodiment, the resorcinarene bowl-shape is built up with imides that allow hydrogen bonding in a self-complementary sense (see Körmer, et. al, Chemistry, a European Journal, 1999, 6:187-195, the entire contents of which are incorporated by reference herein). When the hydrogen bonds form, a capsule is created and that capsule can reversibly bind a donor/acceptor fluorophore.

A wide variety of fluorophores is contemplated for use in the practice of the present invention, such as, for example, xanthenes (e.g., fluoresceins, rhodamines), coumarins (e.g., umbelliferone), benzimides, phenanthridines (e.g., Texas Red), ethidium fluorophores, acridines, cyanines, phthalocyanines, squarines, carbazoles, phenoxazines, porphyrins, quinolines, and the like. In a preferred aspect, the fluorophores are xanthenes or coumarins. See, e.g., Handbook of Fluorescent Probes and Research Products, Eighth Ed., 2001, Molecular Probes, Inc., which is hereby incorporated by reference in its entirety. The fluorophores may absorb in the ultraviolet, visible, or infrared ranges of the electromagnetic spectrum.

Many factors influence the intensity of a signal produced by a fluorescence resonance energy transfer label. For example, the donor fluorophore is chosen so that it has a strong coefficient of molar absorptivity (E) at the chosen excitation wavelength. The acceptor fluorophore should be able to receive energy from the donor fluorophore and in turn, emit radiation at a wavelength different from the excitation wavelength of the donor fluorophore.

The orientation in space of each fluorophore should maximize energy transfer from donor fluorophore to acceptor fluorophore. Energy transfer is dependent on 1/R⁶, wherein R is the distance between the two fluorophores. In addition, the geometrical orientation of the dipoles of the donor and acceptor fluorophores will affect the efficiency of energy transfer between them. In accordance with the present invention, appropriate spacing between the two fluorophores is provided by suitable choice of support member(s) and three-dimensional macromolecular architecture (e.g., rotaxane, resorcinarene, catenane, carcerand, hemicarcerand, calixarene), to which the fluorophores are either covalently attached or associated non-covalently via steric interactions. Mechanical barriers unique to each macromolecular assembly position the fluorophores properly to allow FRET to take place. Thus, the relative orientation of each fluorophore can be varied to optimize the signal produced by invention energy transfer labels during the FRET process. Thus, in accordance with this aspect of the invention, there is provided a method for increasing the intensity of a fluorescence resonance energy transfer signal comprising contacting an analyte with an invention energy transfer label under conditions suitable to form a covalent bond between said analyte and said energy transfer label, thereby forming a labeled analyte, irradiating the analyte at a first wavelength, and detecting energy emission at wavelengths other than the first wavelength.

In accordance with another aspect of the invention, there are provided methods for labeling a biomolecule comprising contacting the biomolecule with an energy transfer label under conditions suitable to form a covalent bond between the biomolecule and the energy transfer label, thereby forming a labeled biomolecule, wherein the energy transfer label comprises at least one donor fluorophore covalently attached to a first support member and at least one acceptor fluorophore covalently attached to a second support member, wherein steric interactions between the support members mechanically link the donor fluorophore and the acceptor fluorophore. Functional groups useful for attaching an energy transfer label to a biomolecule include, for example, hydroxyl, carboxyl, amino, amido, and thio.

Invention fluorescence energy transfer labels may be attached to a wide variety of biomolecules to form bioconjugates. Biomolecules contemplated for use as components of bioconjugates include, for example, nucleosides, nucleotides, oligonucleotides, polynucleotides, polypeptides, and polysaccharides. In one aspect, the biomolecule is preferably an oligonucleotide or a polynucleotide. Energy transfer labels may be attached to oligonucleotides at the 5′-terminus, the 3′-terminus, or on the phosphodiester backbone. Bioconjugates are useful in applications such as, for example, oligonucleotide hybridization probes, PCR primers, and DNA sequencing. See, e.g., U.S. Pat. Nos. 6,255,476; 6,258,544; 6,268,146; 6,270,973; 5,861,287; 5,707,804; 6,207,421; and 6,306,597, each of which is hereby incorporated by reference in their entirety.

Fluorescence energy transfer labels are suitable for use in a wide variety of applications, both qualitative and quantitative. For example, energy transfer labels are suitable for identifying nucleic acids in a multi-nucleic acid mixture. In particular, energy transfer labels are useful in DNA sequencing. DNA sequencing involves extension and termination reactions of oligonucleotide primers. Included as components of the extension and termination reactions are deoxynucleoside triphosphates (dNTP's) and dideoxynucleoside triphosphates (ddNTP's); dNTP's are used to extend the primer and ddNTP's terminate further extension of the primer. The different termination products that are formed are separated and analyzed in order to determine the positioning of the various nucleosides.

Fluorescence energy transfer labels may be used to label oligonucleotide primers, dNTP's, or ddNTP's. Thus, in another aspect of the invention there are provided methods for DNA primer sequencing and DNA terminator sequencing. In DNA primer sequencing, the fluorescence energy transfer label is attached to the primer being extended. Four separate extension/termination reactions are then carried out simultaneously, each extension reaction containing a different ddNTP to terminate the extension reaction. After termination, the reaction products are separated by gel electrophoresis and analyzed. Thus, in this aspect of the invention, there is provided a method for sequencing a polynucleotide comprising forming a mixture of extended labeled primers by hybridizing a polynucleotide with an oligonucleotide primer labeled with an invention energy transfer label in the presence of deoxynucleoside triphosphates, at least one dideoxynucleoside triphosphate, and a DNA polymerase, wherein the DNA polymerase extends the primer with the deoxynucleoside triphosphates until a dideoxynucleoside triphosphate is incorporated which terminates extension of the primer, separating the mixture of extended labeled primers, and determining the sequence of the polynucleotide by irradiating the mixture of extended labeled primers.

In DNA terminator sequencing, the fluorescence energy transfer label is attached to each of the ddNTP's. The extension reaction is performed using deoxynucleoside triphosphates until the labeled ddNTP is incorporated into the extended primer, thus preventing further extension of the primer. The reaction products for each ddNTP are separated and detected. In one aspect, separate extension/termination reactions are conducted for each of the four ddNTP's. In another aspect, a single extension/termination reaction is carried out which contains four different ddNTP's, each labeled with a spectroscopically resolvable invention fluorescence energy transfer label. Thus, in this aspect of the invention, there is provided a method for sequencing a polynucleotide comprising forming a mixture of extended primers by hybridizing a polynucleotide with an oligonucleotide primer in the presence of deoxynucleoside triphosphates, at least one dideoxynucleoside triphosphate labeled with an invention energy transfer label, and a DNA polymerase, wherein the DNA polymerase extends the primer with the deoxynucleoside triphosphates until a labeled dideoxynucleoside triphosphate is incorporated which terminates extension of the primer, separating the mixture of extended primers, and determining the sequence of the polynucleotide by detecting the labeled dideoxynucleoside triphosphate attached to the extended primers.

In the above described sequencing methods, the labeled oligonucleotides are typically separated by electrophoresis, as described in, for example, Rickwood and Hames, Eds., Gel Electrophoresis of Nucleic Acids: A Practical Approach, IRL Press limited, London, 1981. After separation, the labeled oligonucleotides are detected by measuring fluorescence emission from the labeled oligonucleotides after excitation by a standard source, such as, for example, mercury vapor lamp, laser.

The invention will now be described in greater detail by reference to the following non-limiting examples.

### EXAMPLES

Analyses of biomolecules are performed using the methods disclosed in U.S. Pat. Nos. 5,800,996 and 5,688,648, except that fluorescent energy transfer labels having mechanically linked fluorophores are employed.

All target compounds and intermediates described below were characterized using the following techniques. ¹H NMR (600 MHz) and ¹³C NMR (151 MHz) spectra were recorded on a Bruker DRX-600 spectrometer. Matrix-assisted laser desorption/ionization (MALDI) FTMS experiments were recorded on an IonSpec FTMS mass spectrometer. Dichloromethane and THF were passed through columns of activated aluminum oxide as described by Grubbs and coworkers prior to use (D. T. B. Hannah, et al., J. Mater. Chem. 1997, 7, 1985). Coumarin laser fluorophores 2 and 343 were purchased from Acros Organics (Pittsburgh, Pa.). All other reagents were purchased from Sigma-Aldrich (Milwaukee, Wis.) and were used without further purification. Unless otherwise stated, all reactions were performed under an anhydrous nitrogen atmosphere.

### Example 1

### Synthesis of a Rotaxane Energy Transfer Label

A first-generation model rotaxane type fluorescence resonance energy transfer label was synthesized using a strategy introduced by Vögtle (see, Hübner, et al., Angew. Chem. Int. Ed. 1999, 38, 383-386; and Vögtle, et al., Liebigs Ann. 1995, 739-743, the entire contents of which are incorporated herein). In this methodology, an amide “wheel” acts as a template for the reaction between the “axle” and the “stopper”.

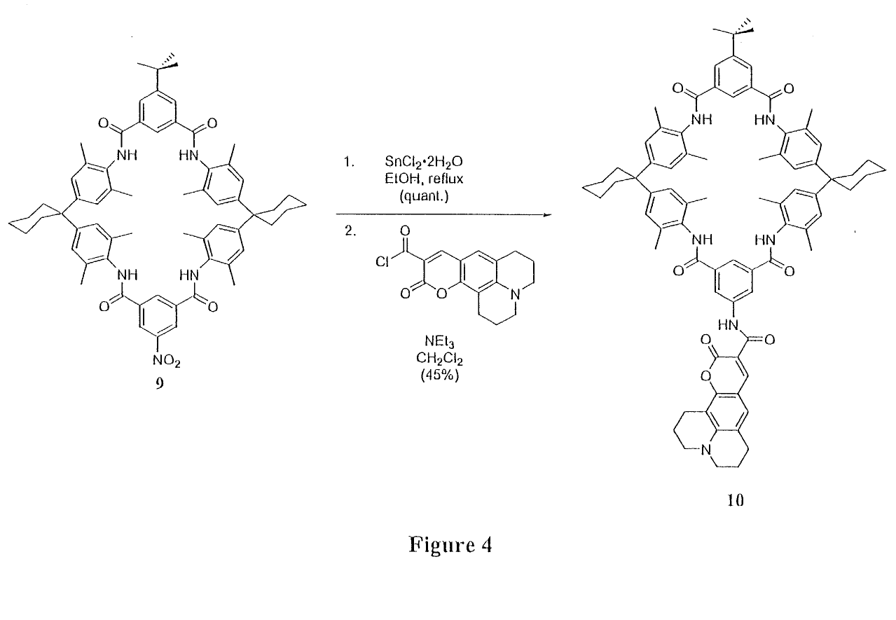

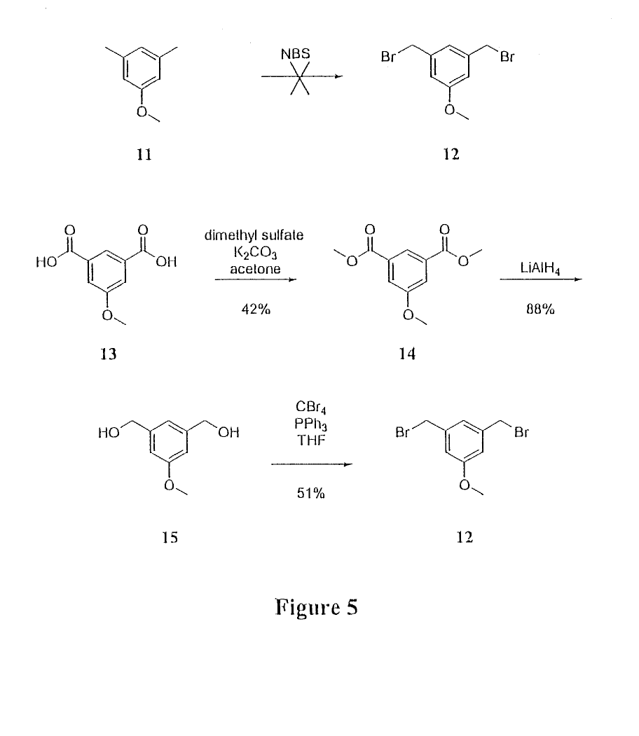

Referring to FIGS. 3 and 4, exemplary macrocycle 9 was synthesized according to the procedure of Hunter (C. Hunter, J. Am. Chem. Soc. 1992, 114, 5303-5311; F. Vögtle, et al., Liebigs Ann. 1996, 921-926; R. Schmieder, et al., Eur. J. Org. Chem. 1998, 2003-2007; C. Reuter, et al., Chem. Eur. J. 1999, 5, 2692-2697; and C. Heim, et al., Helv. Chim. Acta 1999, 82, 746-759, the entire contents of each of which are incorporated by reference herein). The nitro group of macrocycle 9 served as a handle by which to attach the desired acceptor fluorophore. Reduction with tin followed by acylation with the acid chloride laser dye afforded exemplary macrocycle 10.

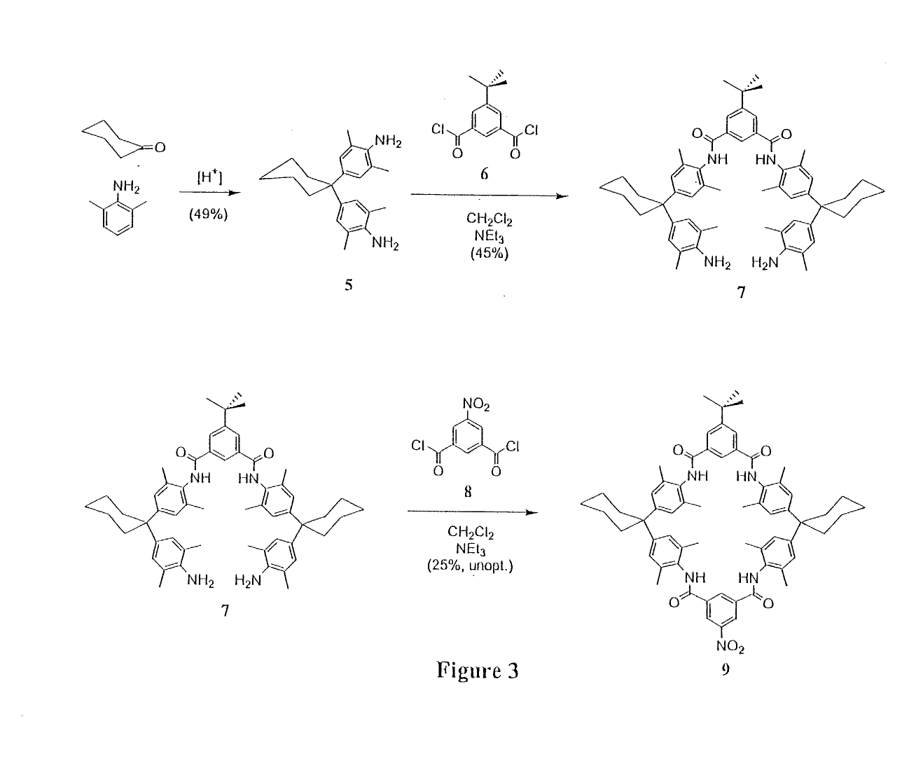

An exemplary linear molecule (“axle”) synthesis (as shown in FIG. 2) is based on a scheme used by Cram and coworkers (D. J. Cram, et al., J. Am. Chem. Soc. 1951, 73, 5691; and H. Steinberg, et al., J. Am. Chem. Soc. 1952, 74, 5388-5391, the entire contents of each of which are incorporated by reference herein).

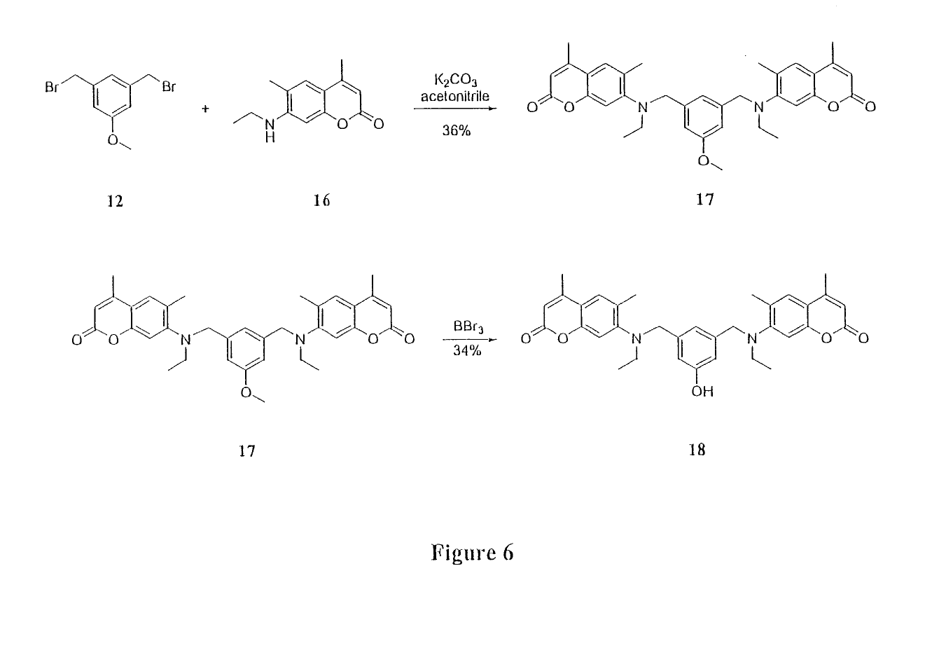

An exemplary stopper molecule was synthesized as described previously (see, for example, S. L. Gilat, et al., J. Org. Chem. 1999, 64, 7474-7484, the entire contents of which are incorporated by reference herein). Referring to FIGS. 5 and 6, generation of dibromide 12 by radical NBS bromination of 3,5-dimethylanisole 11 proved to be a less than ideal synthetic route. As suggested by Bickelhaupt and coworkers, the reaction produces a complex mixture of mono-, di-, and tri-brominated products (G. -J. Gruter, G. -J., et al., J. Org. Chem. 1994, 59, 4473-4481, the entire contents of which are incorporated by reference herein). An alternative route was chosen using 3,5-bis(hydroxymethyl)anisole 15, which was prepared by the method of Raymond and coworkers (T. M. Dewey, et al., Inorg. Chem. 1993, 32, 1792-1738, the entire contents of which are incorporated by reference herein). The conversion of 15 to the dibromide product 12 was accomplished in 51% yield using carbon tetrabromide and triphenylphosphine. The dibromide 12 was then reacted with three equivalents of coumarin 2 16 (S. L. Gilat, et al., J. Org. Chem. 1999, 64, 7474-7484, the entire contents of which are incorporated by reference herein). The stopper 18 was then obtained by phenol deprotection of 17 with boron tribromide in dichloromethane.

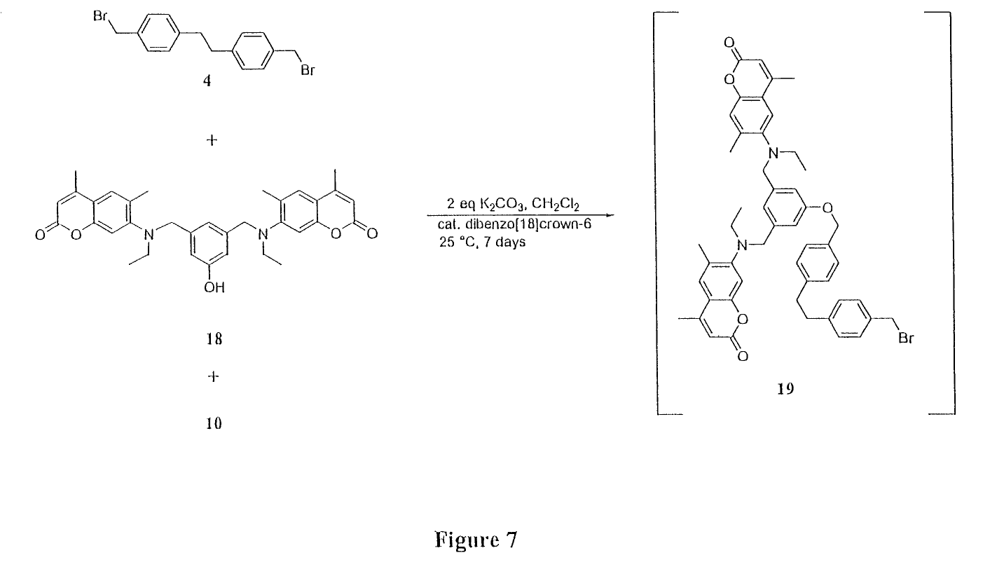

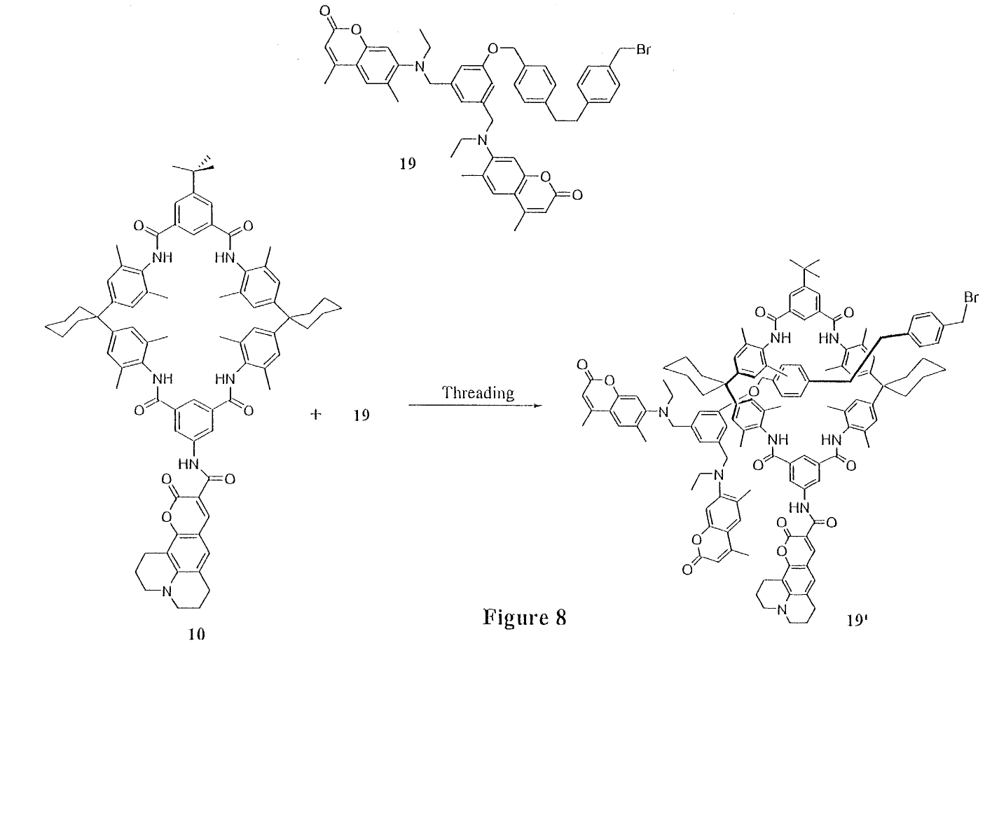

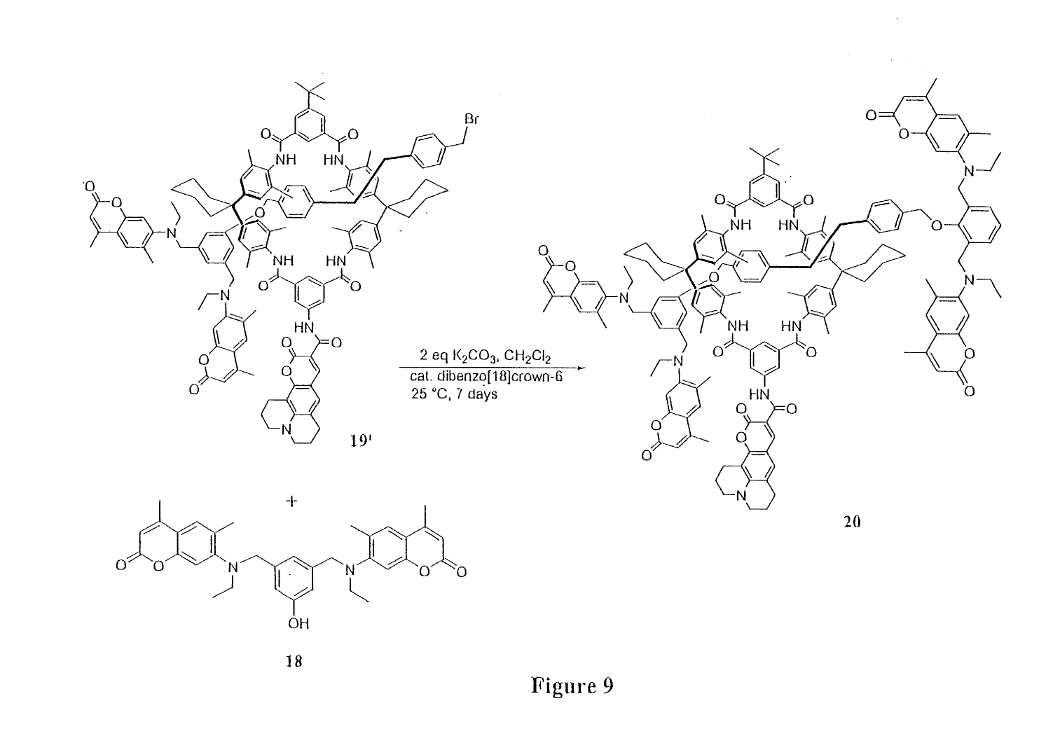

Referring to FIGS. 7, 8, and 9, the rotaxane threading was accomplished by a templation effect. The amide protons of macrocycle 10 served to stabilize the phenoxide ion, which could then displace the benzylic bromide. This reaction occurs first at one end to give intermediate 19 or 19′ and then at the other to give the rotaxane 20. When this reaction occurs at each end of the linear molecule (axle), the threading is complete and the macrocycle (wheel) is locked in place. Reaction under the conditions of Vögtle gave the desired rotaxane as evidenced by ¹H-NMR and fluorescence spectroscopy (vide infra).

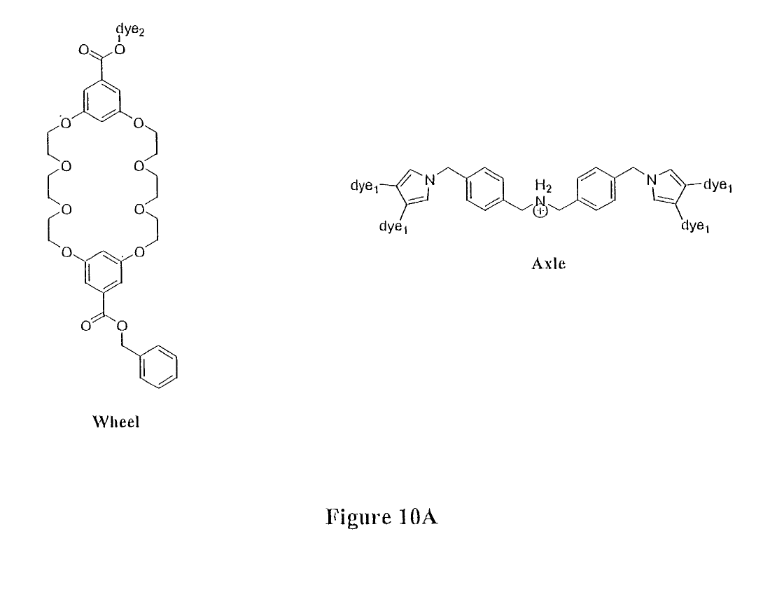

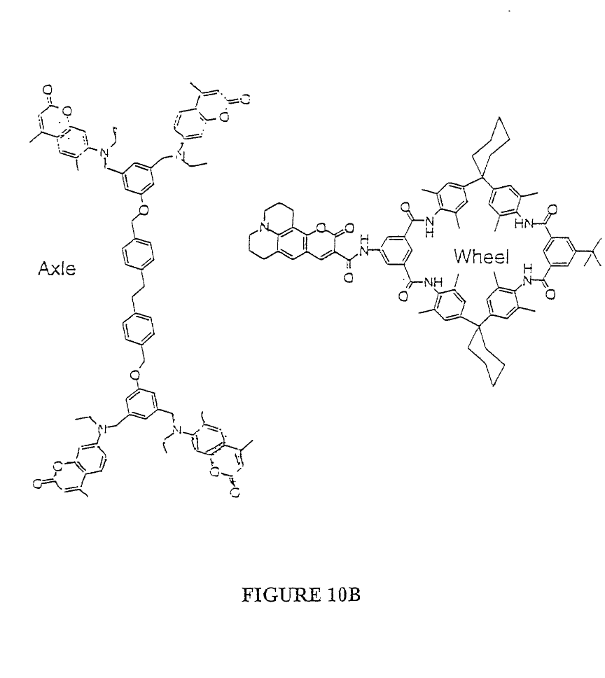

A second generation rotaxane-type energy transfer label is disclosed in FIG. 10A. The rotaxane consists of a dibenzo-crown ether wheel surrounding a linear molecular axle bearing a protonated amine. As in rotaxane 20, two donor fluorophores are attached to each end of the axle. The two esters of the crown ether may be functionalized separately. One is used to attach an acceptor fluorophore and the other is used as a linker to a biomolecule, such as, for example, a dideoxynucleoside (for Sanger DNA sequencing).

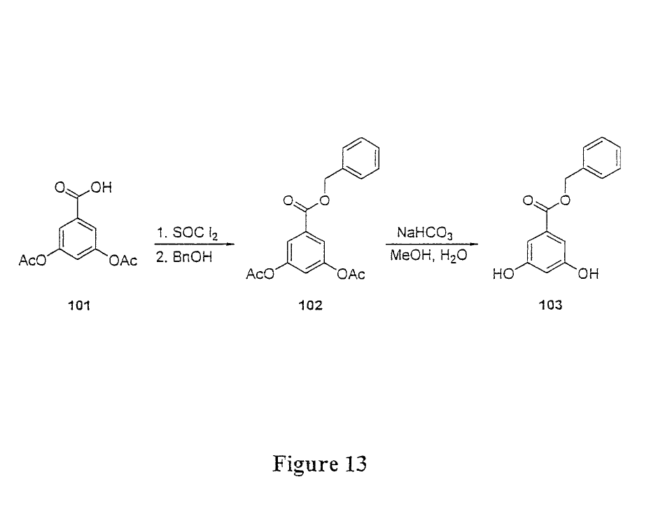

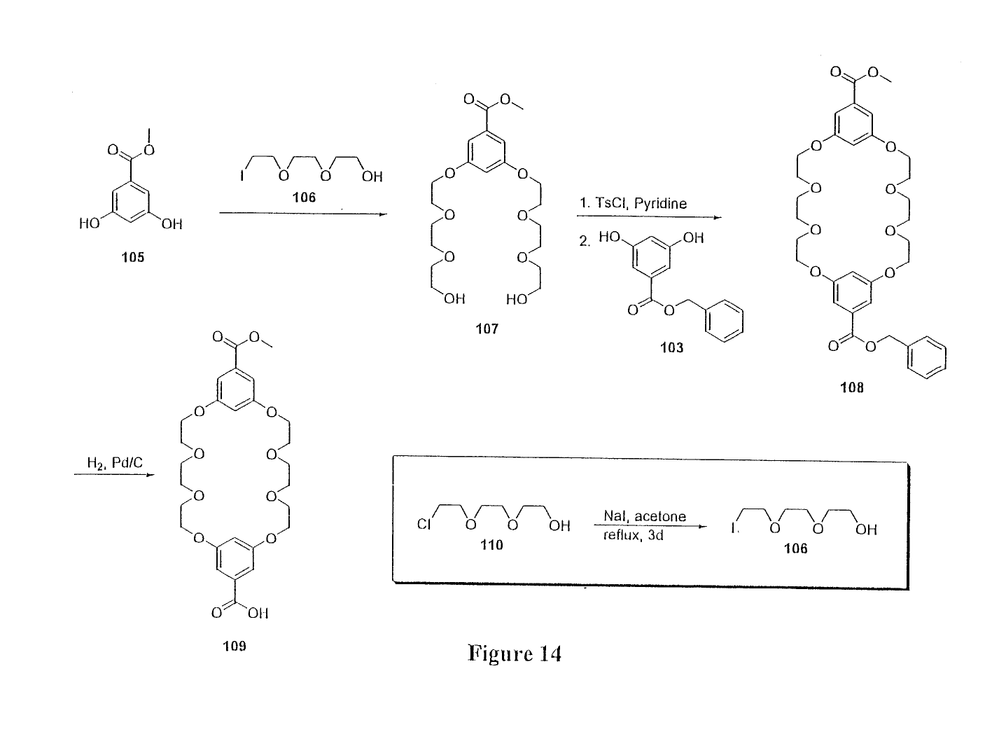

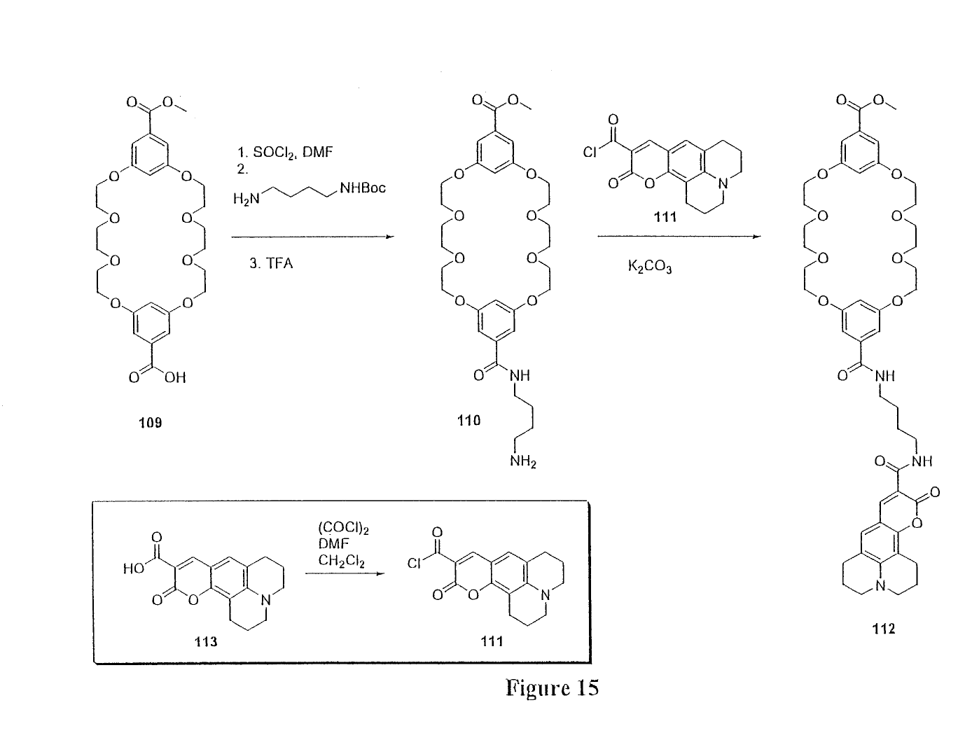

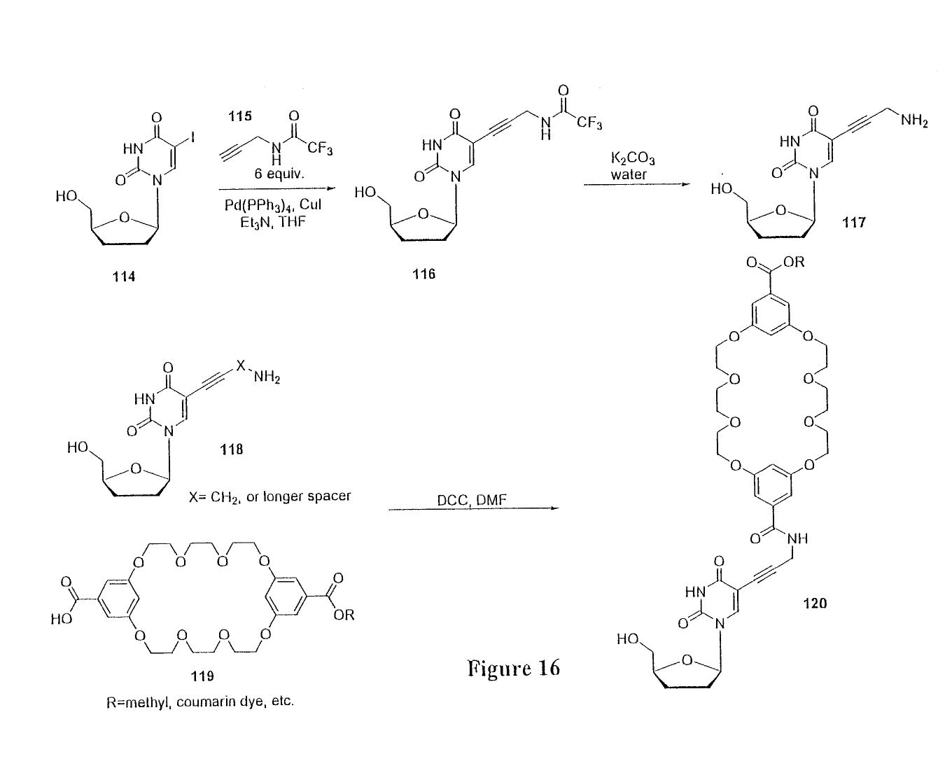

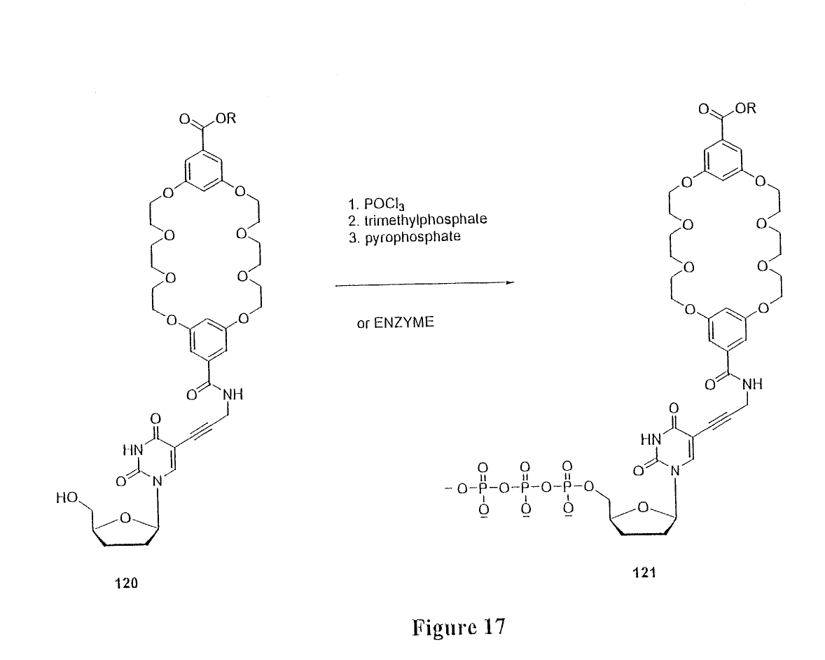

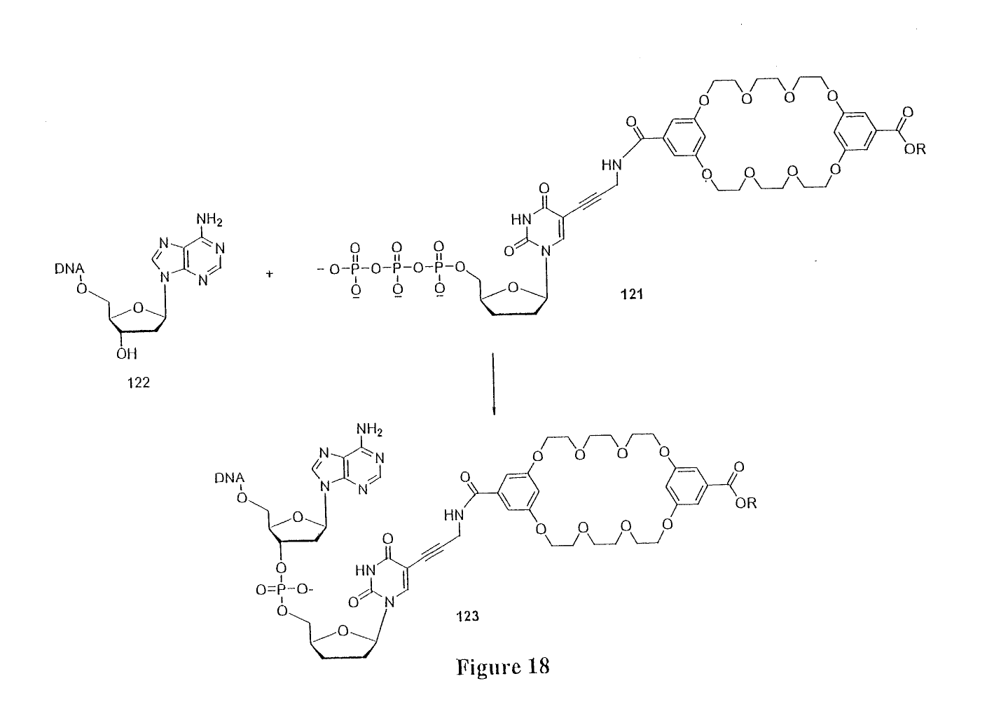

A synthetic scheme for making an embodiment employable in Sanger sequencing, specifically one that is attachable to a dideoxy terminator, is illustrated in FIGS. 13-18. Preparation of a wheel component that has two functional sites, one to attach the acceptor fluorophore and one to attach to the dideoxy terminator is schematically illustrated. Fluorescent energy transfer dyes with different acceptor fluorophores may be incorporated during polymerase extension. The resultant labeled polynucleotide extension products may be characterized with regard to their mobility.

Detailed experimental procedures and characterization data for each intermediate in the synthesis of a rotaxane energy transfer label is provided below.

Dimethyl 1,2-bis(4-carboxyphenyl)ethane (2):

Referring to FIG. 2, dimethyl 1,2-bis(4-carboxyphenyl)ethane (2) was synthesized according to the method of D. J. Cram, et al. (J. Am. Chem. Soc. 1951, 73, 5691 and J. Am. Chem. Soc. 1952, 74, 5388-5391). To a solution of dimethyl 4-dicarboxy-trans-stilbene (2.25 g, 7.60 mmol) in THF (100 mL) was added Raney Nickel. The reaction was then allowed to stir under hydrogen gas at atmospheric pressure at room temperature for 4 hours. The reaction mixture was then poured through Celite and concentrated in vacuo to give 2.20 g of the desired product as a white solid (7.38 mmol, 97%). TLC (3:1 hexanes/EtOAc) R_{f}=0.54.

1,2-Bis(4-hydroxymethylphenyl)ethane (3):

Referring to FIG. 2, 1,2-bis(4-hydroxymethylphenyl)ethane (3) was synthesized according to the method of D. J. Cram, et al. (J. Am. Chem. Soc. 1951, 73, 5691 and J. Am. Chem. Soc. 1952, 74, 5388-5391). To a 0 C solution of dimethyl 1,2-bis(4-carboxyphenyl)ethane (2.0 g, 6.7 mmol) in THF (200 mL) was added lithium aluminum hydride (6.4 g, 60 mmol). After gradually warming to room temperature, the reaction was stirred for 5 hours. The reaction was then quenched with 5 mL of water, 5 mL of 15% NaOH, and 16 mL of water. Filtration of aluminum salts and evaporation of the filtrate gave the product as a white solid. Recrystallization from chloroform provided white needles (1.22 g, 5.03 mmol, 75%). TLC (7:1 hexanes/EtOAc) R_{f}=0.36.

1,2-Bis(4-bromomethylphenyl)ethane (4):

Referring to FIG. 2, 1,2-bis(4-bromomethylphenyl)ethane (4) was synthesized according to the method of C. Heim, et al. (Helv. Chim. Acta 1999, 82, 746-759). To round bottom flask containing 1,2-bis-(4-hydroxymethylphenyl)ethane (1.10 g, 4.54 mmol) and carbon tetrabromide (7.60 g, 22.9 mmol) in THF (100 mL) was slowly added triphenylphosphine (5.94 g, 22.6 mmol). The reaction was covered with aluminum foil and was allowed to stir at room temperature overnight. Filtration through Celite, evaporation, and flash chromatography (7:1 hexanes/ethyl acetate) gave the desired product (395 mg, 1.07 mmol, 24%). TLC (7:1 hexanes/EtOAc) R_{f}=0.53.

1,1-Bis(4-amino-3,5-dimethylphenyl)cyclohexane (5):

Referring to FIG. 3, 1,1-bis(4-amino-3,5-dimethylphenyl)cyclohexane (5) was synthesized according to the method of D. T. B. Hannah, et al. (J. Mater. Chem. 1997, 7, 1985). A mixture of 2,6-dimethylaniline (30 mL, 252 mmol), cyclohexanone (12.6 mL, 121 mmol), and concentrated HCl (30 mL) was refluxed for 2 d. The products were dissolved in 500 mL of water. The solution was then made basic by addition of 1 M NaOH and extracted with 1 L of chloroform. The organic phase was concentrated in vacuo and the residue was crystallized from 500 mL of pentane to give 18.5 g (58 mmol, 48%) of the desired product.

5-tert-Butylisophthaloyl chloride (6):

Referring to FIG. 3, 5-tert-Butylisophthaloyl chloride (6) was synthesized according to the method of C. Hunter (J. Am. Chem. Soc. 1992, 114, 5303-5311). To a suspension of 5-tert-butylisophthalic acid (3.0 g, 13.5 mmol) in dry CH₂Cl₂ (75 mL) was added oxalyl chloride (5 mL, 60 mmol) and DMF (cat.). The mixture was heated to reflux and after 30 min, a homogeneous solution resulted. Heating was continued for an additional 1 hour after which the solution was cooled to room temperature. The solvent was removed in vacuo and the crude acid chloride was obtained in quantitative yield and used without further purification.

5-Nitroisophthaloyl chloride (8):

Referring to FIG. 3, 5-nitroisophthaloyl chloride (8) was synthesized according to the method of C. Hunter (J. Am. Chem. Soc. 1992, 114, 5303-5311). To a suspension of 5-nitroisophthalic acid (3.0 g, 14 mmol) in dry dichloromethane (75 mL) was added oxalyl chloride (5 mL, 60 mmol) and DMF (cat.). The mixture was heated to reflux and after 30 min. a homogeneous solution resulted. Heating was continued for an additional 1 hour after which the solution was cooled to room temperature. The solvent was removed in vacuo and the crude acid chloride was obtained in quantitative yield and used without further purification.

N,N′-Bis{4-[1-(4-amino-3,5-dimethylphenyl)cyclohexyl]-2,6-dimethylphenyl}-5-tert-but ylisophthalamide (7)

Referring to FIG. 3, N,N′-Bis{4-[1-(4-amino-3,5-dimethylphenyl)cyclohexyl]-2,6-dimethylphenyl}-5-tert-butylisoph thalamide (7) was prepared as described by Vögtle with slight modifications (F. Vögtle, et al., Liebigs Ann. 1996, 921-926; R. Schmieder, et al., Eur. J. Org. Chem. 1998, 2003-2007; C. Reuter, et al., Chem. Eur. J. 1999, 5, 2692-2697; and C. Heim, et al., Helv. Chim. Acta 1999, 82, 746-759). Namely, the acid chloride 6 (0.92 g, 3.7 mmol) in dichloromethane (50 mL) was added to diamine 5 (5.0 g, 15.5 mmol) and triethylamine (0.7 mL) in dichloromethane (25 mL) over the course of 4 hours. The crude material was purified by column chromatography on SiO₂ using gradient elution 6:1 CHCl₃/EtOAc (4:1 CHCl₃/EtOAc. The desired product was obtained as an oily tan solid 1.39 g (1.68 mmol, 45%).

Nitro macrocycle (9):

Referring to FIG. 3, nitro macrocycle (9) was synthesized according as described by Vögtle with slight modifications (F. Vögtle, et al., Liebigs Ann. 1996, 921-926; R. Schmieder, et al., Eur. J. Org. Chem. 1998, 2003-2007; C. Reuter, et al., Chem. Eur. J. 1999, 5, 2692-2697; and C. Heim, et al., Helv. Chim. Acta 1999, 82, 746-759). Solutions of 5-nitroisophthaloyl chloride (8, 0.21 g, 0.84 mmol, in 120 mL CHCl₃) and the diamine (7, 0.70 g, 0.84 mmol, in 120 mL CHCl₃) were combined, dropwise over 4 hours, into 600 mL of CHCl₃. After stirring for an additional 12 hours the solvent was evaporated and the crude material was initially purified by column chromatography on SiO₂ (3% EtOH/CHCl₃). All nonpolar fractions were combined and evaporated, and the resulting white powder was triturated with THF. The solids were filtered away and the filtrate was subjected to additional column chromatography on SiO₂ (8:1 CH₂Cl₂/EtOAc). Fractions containing the more nonpolar of two products were combined and evaporated to give an oily solid. Trituration with MeOH and re-evaporation gave the desired macrocycle as a white powder (0.21 g, 25%, unoptimized). ¹H NMR (600 MHz, DMF-d₇) δ 9.69 (s, 2H), 9.29 (s, 2H), 9.26 (s, 1H), 8.85 (d, 2H, J=1.1 Hz), 8.69 (s, 1H), 8.21 (s, 2H), 7.23 (s, 4H), 7.20 (s, 4H), 2.48 (m, 8H), 2.15 (s, 12H), 2.14 (s, 12H), 1.63 (m, 8H), 1.52 (m, 4H), 1.41 (s, 9H); ¹³C NMR (151 MHz, DMF-d₇) δ 165.41, 163.19, 152.76, 149.38, 147.50 (m), 137.08, 135.30, 135.23, 135.17, 133.30, 132.79, 128.18, 126.33, 126.19, 125.63, 125.12, 45.20, 34.85, 31.05, 26.54, 23.21, 18.54, 18.51; IR (thin film) 3292, 2934, 2859, 1670, 1635, 1518, 1313, 1253 cm⁻¹; LRMS (ESI; M+H⁺) calculated for C₆₄H₇₂N₅O₆ 1006.5, found 1006.6.

Amino macrocycle:

Referring to FIG. 4, the amino macrocycle was synthesized according to a general reduction procedure described by D. J. Cram, et al. (J. Am. Chem. Soc. 1992, 114, 7748). To a suspension of the nitro macrocycle 9 (0.050 g, 0.050 mmol) in EtOH (10 mL) was added SnCl₂2H₂O (0.045 g, 0.20 mmol). The mixture was heated to 80 C. for 1 hour prior to the addition of conc. HCl (1.5 mL), which gave a homogeneous solution. After an additional 2 hours the solution was cooled to room temperature and the solvent was evaporated. The residue was suspended in H₂O (10 mL), made strongly basic with 1 M NaOH, and extracted with CHCl₃ (3×10 mL). After drying the combined organic extracts over MgSO₄ and concentration, the amine was isolated quantitatively as a white, oily solid. ¹H NMR (600 MHz, DMF-d₇) δ 9.33 (s, 2H), 9.02 (s, 2H), 8.71 (s, 1H), 8.20 (d, 2H, J=1.0 Hz), 7.97 (s, 1H), 7.45 (d, 2H, J=1.0 Hz), 7.21 (s, 4H), 7.18 (s, 4H), 5.78 (bs, 2H), 2.46 (m, 8H), 2.18 (s, 12H), 2.17 (s, 12H), 1.64 1.61 (m, 8H), 1.54 1.51 (m, 4H), 1.41 (s, 9H); ¹³C NMR (151 MHz, DMF-d₇) δ 165.71, 165.42, 152.74, 150.45, 147.40 (m),136.22, 135.36, 135.28, 135.23, 133.35, 133.29, 128.16, 126.17, 126.13, 125.22, 116.35, 114.80, 45.13,32.14, 31.05, 26.55, 23.21, 18.55, 18.53; IR (thin film) 3336, 2934, 2859, 1661, 1635, 1596, 1514, 1454, 1336, 1253 cm⁻¹; HRMS (MALDI-FTMS; M+Na⁺) calculated for C₆₄H₇₃N₅O₄Na 998.5555, found 998.5527.

Macrocycle (10):

Referring to FIG. 4, to a solution of coumarin 343 (14 mg, 0.050 mmol) in CH₂Cl₂ (10 mL) was added oxalyl chloride (9 μL, 0.10 mmol) and DMF (cat.). After 1 hour at room temperature the solvent was removed and the acid chloride was dried under high vacuum for 1 hour. The material was redissolved in CH₂Cl₂ (7 mL) and treated with a solution of the amine (49 mg, 0.050 mmol) in CH₂Cl₂ (5 mL) and triethylamine (10 μL, 0.075 mmol). The solution was stirred at room temperature for 4 hours. After the solvent was removed, the crude material was purified by column chromatography on SiO₂ (3:1 CHCl₃/EtOAc). The acceptor wheel was isolated as a yellow powder (28 mg, 45%). ¹H NMR (600 MHz, DMF-d₇/CDCl₃) δ 11.24 (s, 1H), 9.30 (s, 2H), 9.27 (s, 2H), 8.73 (s, 1H), 8.68 (s, 1H), 8.54 (s, 1H), 8.50 (s, 2H), 8.19 (d, 2H, J=0.8 Hz), 7.33 (s, 1H), 7.20 (s, 8H), 3.43 (m, 4H), 2.85 (m, 2H), 2.81 (m, 2H), 2.45 (m, 8H), 2.19 (s, 12H), 2.17 (s, 12H), 1.99 1.95 (m, 4H), 1.63 (m, 8H), 1.52 (m, 4H), 1.41 (s, 9H); IR (thin film) 3274, 2931, 2857, 1668, 1634, 1515, 1444, 1309, 1254, 1201, 1172 cm⁻¹.

Dimethyl 5-methoxyisophthalate (14):

Referring to FIG. 5, dimethyl 5-methoxyisophthalate (14) was synthesized according to a method described by T. M. Dewey, et al. (Inorg. Chem. 1993, 32, 1792-1738). Ground anhydrous potassium carbonate (48.6 g, 351 mmol) was added to a solution of 5-methoxyisophthalic acid (20.0 g, 106 mmol) in acetone (200 mL). Dimethyl sulfate was (33.2 mL, 350 mmol) then added via syringe. The reaction was heated to reflux and was allowed to stir for 12 hours, then quenched with a solution of 15% aqueous KOH. After stirring at reflux for an additional 4 hours, the reaction was then cooled, filtered, and evaporated to provide a white solid. Recrystallization from methanol/water gave 10.13 g of the desired product (45 mmol, 42%). ¹H NMR (CDCl₃) (8.28 (s, 1H), 7.75 (s, 2H), 3.94 (s, 6H), 3.90 (s, 3H).

3,5-Bis(hydroxymethyl)anisole (15):

Referring to FIG. 5, 3,5-bis(hydroxymethyl)anisole (15) was synthesized according to a method described by A. B. Pangborn, et al., (Organometallics 1996, 15, 1518-1520). A solution of dimethyl 5-methoxyisophthalate (7.0 g, 31 mmol) in THF was added to a suspension of lithium aluminum hydride (6.0 g, 158 mmol) at 0(C. The reaction was maintained at room temperature for 30 min. The reaction was then quenched with 7 mL of water, 7 mL of 15% NaOH, and 30 mL of water. Filtration of aluminum salts and evaporation of the filtrate gave the product as a white solid. Recrystallization from chloroform provided white needles (4.61 g, 27.4 mmol, 88%). ¹H NMR (CDCl₃) (6.90 (s, 1H), 6.81 (s, 2H), 4.62 (s, 4H), 3.79 (s, 3H).

3,5-Bis(bromomethyl)anisole (12):

Referring to FIG. 5, 3,5-bis(bromomethyl)anisole (12) was synthesized according to the method of S. L. Gilat, et al. (J. Org. Chem. 1999, 64, 7474-7484). To a solution of 3,5-(bishydroxymethyl)anisole (2.00 g, 11.9 mmol) and carbon tetrabromide (8.20 g, 24.7 mmol) in 150 mL THF at 0 C. was added triphenylphosphine (6.55 g, 24.9 mmol). The reaction was allowed to slowly warm to room temperature and to continue to stir overnight. The crude reaction mixture was filtered through Celite and concentrated to give a reddish-orange crystalline precipitate. The desired product was isolated by flash chromatography (9:1 hexanes/chloroform) as a white solid (1.79 g, 6.08 mmol, 51%). TLC (10:1 hexanes/dichloromethane) R_{f}=0.54. ¹H NMR (CDCl₃) (6.98 (s, 1H), 6.84 (s, 2H), 4.42 (s, 4H), 3.80 (s, 3H); ¹³C NMR (CDCl₃) (160.4, 140.0, 122.3, 115.1, 55.9, 33.3.

3,5-Bis(N-(4,6-dimethyl-7-ethylaminocoumarin)methyl)anisole (17):

Referring to FIG. 6, 3,5-bis(N-(4,6-dimethyl-7-ethylaminocoumarin)methyl)anisole (17) was synthesized according to the method of S. L. Gilat, et al. (J. Org. Chem. 1999, 64, 7474-7484). To a solution of 3,5-bis(bromomethyl)anisole (12, 500 mg, 1.7 mmol) in acetonitrile (20 mL) was slowly added an acetonitrile solution of 4,6-dimethyl-7-ethylaminocoumarin (coumarin 2, 16) (1.10 g, 5.1 mmol) and potassium carbonate (2.11 g, 15.3 mmol). The reaction was heated to reflux and continued to stir for 4 days. The solution was allowed to cool to room temperature and filtered. The filtrate was evaporated to dryness in vacuo. Flash chromatography (silica gel, 20:1 dichloromethane/ethyl acetate) provided the desired product as a crystalline solid (400 mg, 0.62 mmol, 36%). TLC (10:1 hexanes/ethyl acetate) R_{f}=0.46.

3,5-Bis(N-(4,6-dimethyl-7-ethylaminocoumarin)methyl)phenol (18):

Referring to FIG. 6, 3,5-bis(N-(4,6-dimethyl-7-ethylaminocoumarin)methyl)phenol (18) was synthesized according to the method of S. L. Gilat, et al. (J. Org. Chem. 1999, 64, 7474-7484). To a dichloromethane solution (10 mL) of 3,5-bis(N-(4,6-dimethyl-7-ethylaminocoumarin)methyl)anisole (17) (188 mg, 0.31 mmol) at 0 C. was slowly added boron tribromide (0.3 mL, 3.1 mmol) in dichloromethane (10 mL). After stirring at room temperature for 2 hours, the crude reaction was poured over crushed ice. The organic phase was separated, washed with aqueous NaHCO₃ and water, and dried over sodium sulfate. This was concentrated to afford a yellow-brown powder. Flash chromatography (7:1 dichloromethane/ethyl acetate) from ethyl acetate gave the product as a yellow powder (58 g, 0.10 mmol, 34%). TLC (7:1 dichloromethane/ethyl acetate) R_{f}=0.19. ¹H NMR (CDCl₃) (7.36 (s, 2 H), 6.90 (s, 2H), 6.77 (s, 1H), 6.74 (s, 2 H), 6.14 (d, J=1.0 Hz, 2 H), 4.18 (s, 4 H), 3.12 (q, J=7.0 Hz, 4 H), 2.40 (m, 12 H), 1.06 (t, J=7.0 Hz, 6 H).

Amide-Based Rotaxane (20)

Referring to FIGS. 7-9, amide-based rotaxane 20 was synthesized according as described by Vögtle with slight modifications (F. Vögtle, et al., Liebigs Ann. 1996, 921-926; R. Schmieder, et al., Eur. J. Org. Chem. 1998, 2003-2007; C. Reuter, et al., Chem. Eur. J. 1999, 5, 2692-2697; and C. Heim, et al., Helv. Chim. Acta 1999, 82, 746-759). To a stirring solution of phenol 18 (18 mg, 0.032 mmol) and potassium carbonate (8.0 mg, 0.058 mmol) in dichloromethane (4 mL) was added dibenzo[18]crown-6 (2 mg) followed by wheel 10 (20 mg, 0.016 mmol). Once the wheel had completely dissolved, 1,2-bis(4-bromomethylphenyl)ethane (4) (59 mg) was added in an additional 2 mL dichloromethane. After stirring at room temperature for 7 days, the crude reaction mixture was concentrated in vacuo and purified by semi-preparative-scale reverse phase-HPLC to give the desired product as a deep yellow solid. ¹H NMR (CDCl₃) (11.10 (s), 8.53 (s), 8.31 (br s), 8.29, (s), 8.11 (br s), 7.32-7.24 (m), 6.09 (s), 4.90 (br s), 4.04 (s), 3.35 (s), 2.95 (br s), 2.35-2.25 (m), 2.14(s), 1.63 (s), 1.49 (s), 1.21 (s), 0.84 (s).

### Example 2

### Synthesis of a Catenane Energy Transfer Label

Referring to FIG. 12B, a solution of Cu(MeCN)₄PF₆ (142 mg, 0.380 mmol) in degassed acetonitrile (60 ml) was added to a stirred solution of macrocycle 34 (232 mg, 0.345 mmol) in CH₂Cl₂ (60 ml) at room temperature under argon. After stirring the solution for 30 min, a solution of thread 35 (263 mg, 0.345 mmol) in CH₂Cl₂ (60 ml) was added, and the stirring was continued for 2 h under argon at room temperature. The solvents were removed under reduced pressure to leave a dark brown solid of precatenate 41. This compound was used without further purification. ¹H NMR (DMSO-d₆) δ 3.39 (t, J=6.4 Hz, 4H), 3.57-3.63 (m, 8H), 3.65-3.69 (m, 4H), 3.70-3.74 (m, 8H), 3.85-3.88 (m, 7H), 4.35-4.39 (m, 4H), 6.03 (d, J=8.6 Hz, 4H), 6.11 (d, J=8.7 Hz, 4H), 7.22 (s, 3H), 7.28 (d, J=8.6 Hz, 4H), 7.45 (d, J=8.7 Hz, 4H), 7.97 (d, J=8.3 Hz, 2H), 8.00 (d, J=8.3 Hz, 2H), 8.10 (s, 2H), 8.17 (s, 2H), 8.60 (d, J=8.3 Hz, 2H), 8.69 (d, J=8.3 Hz, 2H). ESI-MS: [M+H]⁺: expected: 1496; observed: 1496.

To the solution of precatenate 41 in DMF (60 ml) N-Boc-3,5-dihydroxybenzylamine 36 (99 mg, 0.414 mmol), Cu(MeCN)₄PF₆ (129 mg, 0.345 mmol), and L-(+)-ascorbic acid (41 mg, 0.233 mmol) were added. The resulting solution was degassed and added to a suspension of Cs₂CO₃ (1124 mg, 3.45 mmol) in dry degassed DMF (150 ml) over a period of 4 h at 40° C. under Ar in the dark. These conditions were maintained for 1 day, then the mixture was stirred for another two days at 50° C. The reaction mixture was filtered, the solvent was evaporated, and the residue was dissolved in CH₂Cl₂ (30 ml) and water (30 ml). Separation of the phases, the organic layer was dried over MgSO₄, filtered, and concentrated. The residue was dissolved in MeCN (20 ml), and the solution of 500 mg KCN in water (20 ml) was added. Stirring the solution overnight. Evaporation of MeCN, extraction with CHCl₃. The organic phase was dried over MgSO₄, filtered, and concentrated. HPLC-MS analysis (eluent: MeCN+0.05% TFA-H₂O +0.05% TFA, gradient: 0% MeCN-100% MeCN) of the product mixture revealed the presence of catenane 29. Separation by preparative HPLC, conditions: column: βsil C₁₈ preparative column, flow rate: 8 ml/min, solvent A: H₂O/0.05% TFA, solvent B: MeCN/0.05% TFA, gradient: 60% B→100% B (in 7 min)→60% B (in 0.1 min). 125 mg, 26%. ¹H NMR (acetone-d₆) δ 1.39 (s, 9H), 3. 83 (s, 3H), 3.88-3.96 (m, 12H), 4.01-4.04 (m, 4H), 4.05-4.08 (m, 4H), 4.12-4.16 (m, 4H), 4.21 (bs, 2H), 4.31-4.34 (m, 4H), 4.40-4.43 (m, 4H), 6.36 (d, J=7.4 Hz, 4H), 6.65 (d, J=2.2 Hz, 2H), 6.70 (s, 11H), 7.08 (t, J=2.2 Hz, 1H), 7.18 (d, J=8.8 Hz, 2H), 7.27 (d, J=2.2 Hz, 2H), 7.35-7.41 (m, 4H), 7.78-7.84 (m, 4H), 7.92-7.98 (m, 6H), 8.32 (d, J=8.8 Hz, 2H), 8.50-8.54 (m, 2H), 8.54-8.60 (m, 4H). ESI-MS: [M+H]⁺: expected: 1416; observed: 1416.

### Example 3

### Spectroscopic Data for the Rotaxane Energy Transfer Label

Excitation of the donor fluorophore on the rotaxane stopper was expected to result in through-space energy transfer to the acceptor fluorophore located on the wheel. Ideally, no donor emission would be observed in the fluorescence spectrum, with relatively intense emission by the acceptor dye. Due to the strong spatial dependence of energy transfer, the donor and acceptor dyes should not communicate intermolecularly at moderate concentrations.

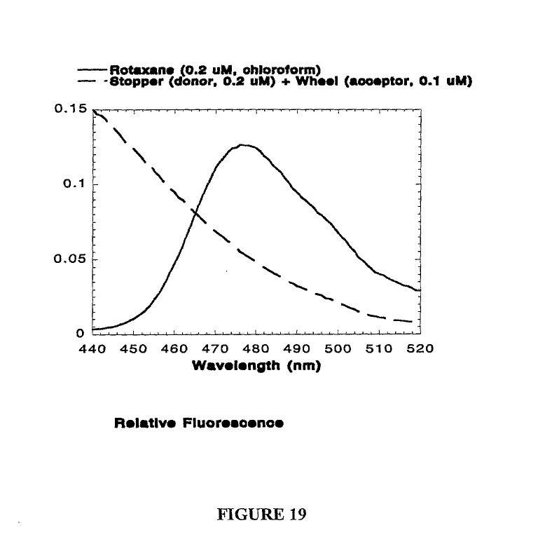

Fluorescence spectra were obtained for four samples in chloroform: (1) stopper 18 (donor, 0.2 (M), (2) wheel 10 (acceptor, 0.1 (M), (3) stopper+wheel, and (4) rotaxane 20 (0.2 (M). Samples 1, 3, and 4 were excited at 340 nm and sample 3 was excited at 430 mn. The spectra of sample 3 (broken line) and sample 4 (solid line) are shown together in Scheme 1 (not normalized). In the mixture of free stopper and wheel, the fluorescence spectrum reflects normal emission by the stopper. The rotaxane fluorescence spectrum showed very different properties. The donor emission was almost completely suppressed and the emission profile reflected that of emission by the acceptor fluorophore (see FIG. 19).

The assembled rotaxane showed very efficient energy transfer from the four donors at the ends of the linear molecule (axle) to the single acceptor on the macrocycle (wheel). These four donors act as light-harvesting dendrimers. The four donors provide a dividend: the system is multifold more sensitive than a fluorescent label having a single simple, covalently-linked energy transfer fluorophore.

Further evidence for structure of rotaxane 20 came through mass spectrometry. MALDI Mass Spectral analysis revealed an [M+Na] peak at 2557. Electrospray mass spectrometry showed peaks at 2557 and 2579 in positive ion mode and at 2591 in negative ion mode. The proton NMR spectrum in chloroform-d was used as further evidence for the rotaxane structure of 20. The amide proton of the 10 shifted from 11.33 ppm to 11.10 ppm. Each of the other wheel protons remained relatively unchanged. The benzylic protons of the axle 4 shifted from 4.49 and 2.90 to and 4.90 and 2.87 ppm respectively, with significant broadening of the former. The phenol proton of stopper 18 (at 5.36 ppm) did not appear in 20. Based on these diagnostic changes, we can reasonably conclude that the structural assignment of 20 is correct.

While the invention has been described in detail with reference to certain preferred embodiments thereof, it will be understood that modifications and variations are within the spirit and scope of that which is described and claimed.

We claim:

1. An energy transfer label comprising at least one donor fluorophore, at least one acceptor fluorophore, and at least one support member, wherein steric interactions between two or more of said donor fluorophore, said acceptor fluorophore, and said support member induce non-covalent association between said donor fluorophore, said acceptor fluorophore, and said support member, thereby forming a macromolecular structure which mechanically links said donor fluorophore and said acceptor fluorophore.

2. An energy transfer label according to claim 1 comprising at least two support members.

3. An energy transfer label according to claim 2 comprising a first support member and a second support member.

4. An energy transfer label according to claim 2, wherein said fluorophores are noncovalently associated with said support members.

5. An energy transfer label according to claim 3, wherein said donor fluorophore is covalently attached to said first fluorophore and said acceptor fluorophore is covalently attached to said second support member.

6. An energy transfer label according to claim 3, wherein said steric interactions physically interlock said first support member with said second support member, thereby mechanically linking said donor fluorophore and said acceptor fluorophore.

7. An energy transfer label according to claim 5, wherein said first support member interacts sterically with said second support member to form a rotaxane.

8. An energy transfer label according to claim 6, wherein said first support member physically interlocks with said second support member to form a catenane.

9. An energy transfer label according to claim 3, wherein said first support member has the structure:
St-L-St,
wherein:
L is hydrocarbyl linking moiety, and
St is a stopper moiety capable of being covalently attached to said linking moiety and at least one donor or acceptor fluorophore.

10. An energy transfer label according to claim 9, wherein said stopper moiety is a substituted cyclic, heterocyclic, aryl, or heteroaryl group.

11. An energy transfer label according to claim 10, wherein said substituents are hydroxyl, amine, carboxyl, amide, hydroxyalkyl, or aminoalkyl.

12. An energy transfer label according to claim 9, wherein said hydrocarbyl linking moiety comprises at least one aryl group.

13. An energy transfer label according to claim 12, wherein said hydrocarbyl linking moiety comprises at least two aryl groups.

14. An energy transfer label according to claim 13, wherein said at least two aryl groups are separated by an optionally substituted alkyl group or heteroalkyl group.

15. An energy transfer label according to claim 14, wherein said optionally substituted alkyl group is a C₁ to about C₆ alkyl group.

16. An energy transfer label according to claim 3, wherein said second support member is a macrocycle, wherein said macrocycle is capable of being covalently attached to at least one donor or acceptor fluorophore and is capable of being covalently attached to a biomolecule.

17. An energy transfer label according to claim 16, wherein said macrocycle comprises moieties selected from optionally substituted alkyl, cycloalkyl, oxyalkyl, aryl, heteroaryl, and heterocyclic.

18. An energy transfer label according to claim 17, wherein said macrocycle comprises optionally substituted aryl groups or heteroaryl groups.

19. An energy transfer label according to claim 18, wherein said aryl or heteroaryl groups are linked via said substituents.

20. An energy transfer label according to claim 19, wherein said substituents are alkyl, amide, carboxyl, hydroxy, hydroxyalkyl, oxyalkyl, amino, or alkylamino.

21. An energy transfer label according to claim 17, wherein said macrocycle comprises optionally substituted oxyalkyl moieties.

22. An energy transfer label according to claim 21, wherein said macrocyclic ring is a crown ether.

23. An energy transfer label according to claim 16, wherein said biomolecule is a nucleoside, nucleotide, oligonucleotide, polynucleotide, protein, or polysaccharide.

24. An energy transfer label according to claim 23, wherein said biomolecule is an oligonucleotide or a polynucleotide.

25. An energy transfer label according to claim 3, wherein said first support member and said second support member are macrocycles.

26. An energy transfer label according to claim 25, wherein said macrocycles are physically interlocked.

27. An energy transfer label according to claim 26, wherein said macrocycles are capable of being covalently attached to at least one donor or acceptor fluorophore and a biomolecule.

28. An energy transfer label according to claim 27, wherein said macrocycles comprise moieties selected from optionally substituted alkyl, cycloalkyl, oxyalkyl, aryl, heteroaryl, and heterocyclic.

29. An energy transfer label according to claim 28, wherein said macrocyclic rings comprise optionally substituted aryl groups or heteroaryl groups.

30. An energy transfer label according to claim 29, wherein said optionally substituted aryl or heteroaryl groups are linked via said substituents.

31. An energy transfer label according to claim 30, wherein said substituents are aalkyl, amide, carboxyl, hydroxy, hydroxyalkyl, oxyalkyl, amino, or alkylamino.

32. An energy transfer label according to claim 1, comprising one support member.

33. An energy transfer label according to claim 32, wherein said support member is a carcerand, hemicarcerand, resorcinarene, or calixarene.

34. An energy transfer label according to claim 1, wherein said fluorophores are xanthenes, coumarins, benzimides, phenanthridines, ethidium fluorophores, acridines, cyanines, phthalocyanines, squarines, carbazoles, phenoxazines, porphyrins, or quinolines.

35. An energy transfer label according to claim 34, wherein said fluorophores are xanthenes or coumarins.

36. An energy transfer label according to claim 35, wherein said fluorophores are xanthenes.

37. An energy transfer label according to claim 36, wherein said fluorophores are fluoresceins or rhodamines.

38. A bioconjugate comprising an energy transfer label according to claim 1 covalently attached to a biomolecule.

39. A bioconjugate according to claim 38 wherein said biomolecule is a nucleoside, nucleotide, oligonucleotide, polynucleotide, polypeptide, or polysaccharide.

40. A bioconjugate according to claim 39 wherein said biomolecule is an oligonucleotide or a polynucleotide.

41. A method for labeling a biomolecule comprising contacting said biomolecule with an energy transfer label under conditions suitable to form a covalent bond between said biomolecule and said energy transfer label according to claim 1, thereby formling a labeled biomolecule.

42. A method for labeling a biomolecule comprising contacting said biomolecule with an energy transfer label, under conditions suitable to form a covalent bond between said biomolecule and said energy transfer label, thereby forming a labeled biomolecule, wherein said energy transfer label comprises at least one donor fluorophore covalently attached to a first support member and at least one acceptor fluorophore covalently attached to a second support member, wherein steric interactions between said support members mechanically link said donor fluorophore and said acceptor fluorophore.

43. A method according to claim 42, wherein said biomolecule is a nucleoside, nucleotide, oligonucleotide, polynucleotide, polypeptide, or polysaccharide.

44. A method according to claim 43, wherein said biomolecule is an oligonucleotide or a polynucleotide.

45. A method for detecting a biomolecule comprising
contacting said biomolecule with an energy transfer label under conditions suitable to form a covalent bond between said biomolecule and said energy transfer label, thereby forming a labeled biomolecule, wherein said energy transfer label comprises at least one donor fluorophore covalently attached to a first support member and at least one acceptor fluorophore covalently attached to a second support member, wherein steric interactions between said support members mechanically link said donor fluorophore and said acceptor fluorophore,
irradiating said labeled biomolecule at a first wavelength, and
detecting energy emission at a second wavelength.

46. A method for identifying nucleic acids in a multi-nucleic acid mixture comprising
contacting said nucleic acids with a plurality of energy transfer labels under conditions suitable to form a covalent bond between said nucleic acids and said energy transfer labels, thereby forming labeled nucleic acids, wherein said energy transfer label comprises at least one donor fluorophore covalently attached to a first support member and at least one acceptor fluorophore covalently attached to a second support member, wherein steric interactions between said support members mechanically link said donor fluorophore and said acceptor fluorophore, and wherein said energy transfer labels comprise donor fluorophores which absorb radiation at a first wavelength and acceptor fluorophores which emit radiation at wavelengths other than said first wavelength,
irradiating said labeled nucleic acids at said first wavelength, and
detecting energy emission at said wavelengths other than said first wavelength.

47. A method for sequencing a polynucleotide comprising
forming a mixture of extended labeled primers by hybridizing a polynucleotide with an oligonucleotide primer labeled with an energy transfer label in the presence of deoxynucleoside triphosphates, at least one dideoxynucleoside triphosphate, and a DNA polymerase, wherein the DNA polymerase extends the primer with the deoxynucleoside triphosphates until a dideoxynucleoside triphosphate is incorporated which terminates extension of the primer, wherein said energy transfer label comprises at least one donor fluorophore covalently attached to a first support member and at least one acceptor fluorophore covalently attached to a second support member, wherein steric interactions between said support members mechanically link said donor fluorophore and said acceptor fluorophore,
separating said mixture of extended labeled primers,
determining the sequence of the polynucleotide by irradiating said mixture of extended labeled primers.

48. A method for sequencing a polynucleotide comprising
forming a mixture of extended primers by hybridizing a polynucleotide with an oligonucleotide primer in the presence of deoxynucleoside triphosphates, at least one dideoxynucleoside triphosphate labeled with an energy transfer label, and a DNA polymerase, wherein the DNA polymerase extends the primer with the deoxynucleoside triphosphates until a labeled dideoxynucleoside triphosphate is incorporated which terminates extension of the primer, wherein said energy transfer label comprises at least one donor fluorophore covalently attached to a first support member and at least one acceptor fluorophore covalently attached to a second support member, wherein steric interactions between said support members mechanically link said donor fluorophore and said acceptor fluorophore,
separating the mixture of extended primers, and
determining the sequence of the polynucleotide by detecting the labeled dideoxynucleoside triphosphate attached to the extended primers.

49. A method for sequencing a polynucleotide comprising
forming a mixture of extended primers by hybridizing a polynucleotide with an oligonucleotide primer in the presence of deoxynucleoside triphosphates labeled with an energy transfer label, at least one dideoxynucleoside triphosphate, and a DNA polymerase, wherein the DNA polymerase extends the primer with the labeled deoxynucleoside triphosphates until a dideoxynucleoside triphosphate is incorporated which terminates extension of the primer, wherein said energy transfer label comprises at least one donor fluorophore covalently attached to a first support member and at least one acceptor fluorophore covalently attached to a second support member, wherein steric interactions between said support members mechanically link said donor fluorophore and said acceptor fluorophore,
separating the mixture of extended primers, and
determining the sequence of the polynucleotide by detecting the labeled deoxynucleoside triphosphates attached to the extended primers.

50. A method for increasing the intensity of a fluorescence resonance energy transfer signal comprising contacting an analyte with an energy transfer label under conditions suitable to form a covalent bond between said analyte and said energy transfer label, wherein said energy transfer label comprises at least one donor fluorophore covalently attached to a first support member and at least one acceptor fluorophore covalently attached to a second support member, wherein steric interactions between said support members mechanically link said donor fluorophore and said acceptor fluorophore, thereby forming a labeled analyte, irradiating said analyte at a first wavelength, and detecting energy emission at wavelengths other than said first wavelength.

51. An energy transfer label comprising a plurality of donor fluorophores, at least one acceptor fluorophore, and at least one support member, wherein steric interactions between two or more of said donor fluorophore, said acceptor fluorophore, and said support member induce non-covalent association between said donor fluorophore, said acceptor fluorophore, and said support member, thereby forming a macromolecular structure which mechanically links said donor fluorophore and said acceptor fluorophore.

52. An energy transfer label comprising at least one donor fluorophore, a plurality of acceptor fluorophores, and at least one support member, wherein steric interactions between two or more of said donor fluorophore, said acceptor fluorophore, and said support member induce non-covalent association between said donor fluorophore, said acceptor fluorophore, and said support member, thereby forming a macromolecular structure which mechanically links said donor fluorophore and said acceptor fluorophore.
